(19) United States
(12) Patent Application Publication
(10) Pub. No.: US 2022/0071267 A1
(43) Pub. Date: Mar. 10, 2022

(54) AEROSOLIZABLE FORMULATION

(71) Applicant: NICOVENTURES TRADING LIMITED, London (GB)

(72) Inventor: Ross CABOT, London (GB)

(21) Appl. No.: 17/290,411

(22) PCT Filed: Oct. 31, 2019
(86) PCT No.: PCT/GB2019/053096

§ 371 Date: Apr. 30, 2021

(30) Foreign Application Priority Data
Nov. 1, 2018 (GB) 1817866.5
May 3, 2019 (GB) 1906242.1

(51) Int. Cl. A24B 15/167; A24B 15/28; A24B 15/34; A24F 40/05; A24F 40/10

(52) U.S. Cl. CPC A24B 15/167; A24B 15/284; A24F 40/10; A24F 40/05; A24B 15/34

(57) ABSTRACT

There is provided an aerosolisable formulation comprising (i) water; (ii) one or more flavours to be encapsulated; (iii) one or more encapsulating materials; wherein in the presence of water the energy of binding of the one or more encapsulating materials with the one or more flavours to be encapsulated is from −0.5 to −8 kcal/mol.

### PRIORITY CLAIM

The present application is a National Phase entry of PCT Application No. PCT/GB2019/053096, filed Oct. 31, 2019 which claims priority from GB Patent Application No. 1906242.1 filed May 3, 2019 and GB Patent Application No. 1817866.5 filed Nov. 1, 2018, each of which is hereby fully incorporated herein by reference.

### FIELD OF THE INVENTION

The present disclosure relates to an aerosolizable formulation, a method of forming the same, a container containing the same, a device containing the same and processes and uses of the same.

### BACKGROUND TO THE INVENTION

Electronic aerosol provision systems such as e-cigarettes generally contain a reservoir of liquid which is to be vaporized, typically containing nicotine. When a user inhales on the device, a heater is activated to vaporize a small amount of liquid, which is therefore inhaled by the user.

The use of e-cigarettes in the UK has grown rapidly, and it has been estimated that there are now over a million people using them in the UK.

One challenge faced in providing such systems is to provide from the aerosol provision device an aerosol to be inhaled which provides consumers with an acceptable experience. Some consumers may prefer an e-cigarette that generates an aerosol that closely ‘mimics’ smoke inhaled from a tobacco product such as a cigarette. Aerosols from e-cigarettes and smoke from tobacco products such as cigarettes provides to the user a complex chain of flavor in the mouth, nicotine absorption in the mouth and throat, followed by nicotine absorption in the lungs. These various aspects are described by users in terms of flavor, intensity/quality, impact, irritation/smoothness and nicotine reward. Nicotine contributes to a number of these factors, and is strongly associated with factors such as impact, irritation and smoothness; these are readily perceived by consumers, and e-cigarettes may offer too much or too little of these parameters for consumers, depending upon individual preferences. Nicotine reward is particularly complex as it results from both the amount of and speed with which nicotine is absorbed from the lining of the mouth, this is typically nicotine in the vapor phase, and from the amount and speed nicotine that is absorbed from the lungs, this is typically nicotine in the particulate phase of the aerosol which is inhaled. Each of these factors, and their balance, can strongly contribute to consumer acceptability of an e-cigarette. Providing means to optimize the overall vaping experience is therefore desirable to e-cigarette manufacturers.

A further challenge facing such systems is the continued demand for harm reduction. Harm from cigarette and e-cigarette devices primarily comes from toxicants. Therefore, there is a desire to reduce or remove the components which may form toxicants.

### SUMMARY OF THE INVENTION

In one aspect there is provided an aerosolizable formulation comprising

(i) water

(ii) one or more flavors to be encapsulated;

(iii) one or more encapsulating materials; and

(iv) nicotine;

wherein in the presence of water the energy of binding of the one or more encapsulating materials with the one or more flavors to be encapsulated is from −0.5 to −8 kcal/mol.

In one aspect there is provided an aerosolizable formulation comprising

(i) water

(ii) one or more flavors to be encapsulated; and

(iii) one or more encapsulating materials having a solubility in water of at least 50% of the solubility in water of the one or more flavors to be encapsulated; and

(iv) nicotine.

In one aspect there is provided a process for forming an aerosol, the process comprising aerosolizing an aerosolizable formulation comprising
(i) water
(ii) one or more flavors to be encapsulated; and
(iii) one or more encapsulating materials;

wherein in the presence of water the energy of binding of the one or more encapsulating materials with the one or more flavors to be encapsulated is from −0.5 to −8 kcal/mol.

In one aspect there is provided a contained aerosolizable formulation comprising

(a) a container; and

(b) an aerosolizable formulation comprising
(i) water
(ii) one or more flavors to be encapsulated;
(iii) one or more encapsulating materials; and
(iv) nicotine;

wherein in the presence of water the energy of binding of the one or more encapsulating materials with the one or more flavors to be encapsulated is from −0.5 to −8 kcal/mol.

In one aspect there is provided an electronic aerosol provision system comprising:

(a) an aerosolizer for aerosolizing formulation for inhalation by a user of the electronic aerosol provision system;

(b) a power supply comprising a cell or battery for supplying power to the aerosolizer

(c) an aerosolizable formulation comprising
(i) water
(ii) one or more flavors to be encapsulated; and
(iii) one or more encapsulating materials;

wherein in the presence of water the energy of binding of the one or more encapsulating materials with the one or more flavors to be encapsulated is from −0.5 to −8 kcal/mol.

In one aspect there is provided a process for improving the sensory properties of an aerosolized formulation, the process comprising the steps of aerosolizing an aerosolizable formulation comprising
(i) water;
(ii) one or more flavors to be encapsulated; and
(iii) one or more encapsulating materials;

wherein in the presence of water the energy of binding of the one or more encapsulating materials with the one or more flavors to be encapsulated is from −0.5 to −8 kcal/mol.

### BRIEF DESCRIPTION OF THE DRAWINGS

The present disclosure will now be described in further detail by way of example only with reference to the accompanying figure in which:

FIG. 1 shows a graph illustrating variation of pₛKₐ₂ with nicotine concentration;

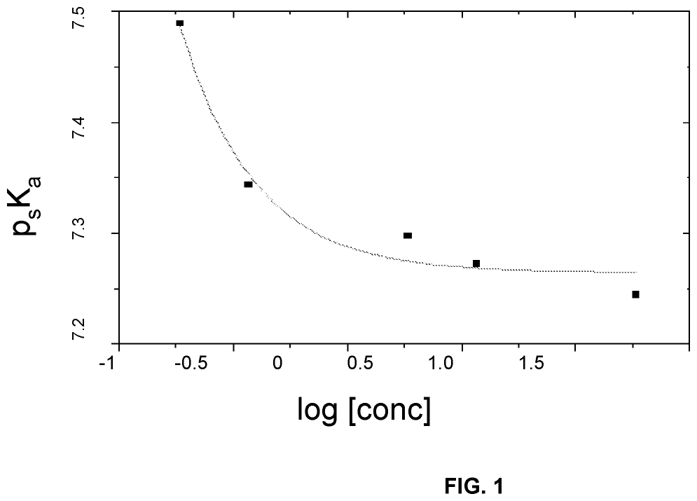

FIG. 2 shows a docking schematic for unsubstituted (β)-cyclodextrin;

FIG. 3 shows a docking schematic for Hydroxypropyl-(β)-cyclodextrin with substitution at 7 sites; and

FIG. 4 shows a docking schematic for Hydroxypropyl-(β)-cyclodextrin with substitution at 5 sites.

### DETAILED DESCRIPTION

As discussed herein in one aspect there is provided an aerosolizable formulation comprising (i) water; (ii) one or more flavors to be encapsulated; (iii) one or more encapsulating materials; and (iv) nicotine; wherein in the presence of water the energy of binding of the one or more encapsulating materials with the one or more flavors to be encapsulated is from −0.5 to −8 kcal/mol.

We have found that an advantageous system may be provided in which, in the presence of water, the energy of binding of the one or more encapsulating materials with the one or more flavors to be encapsulated is from −0.5 to −8 kcal/mol. We have found that by selection of one or more encapsulating materials together with selection of the one or more flavors to be encapsulated such that the two materials have the required energy of binding an advantageous flavordelivery system is provided. In particular, the flavor delivery system binds the flavor strongly enough for it to be delivered in use but not so strongly that it will not dissociate in use from the encapsulating material. Thus a flavor may be stably delivered whilst still providing a strong flavor release for the end user.

As is understood by one skilled in the art, nicotine may exist in unprotonated form, monoprotonated form or diprotonated form. The structures of each of these forms are given below.

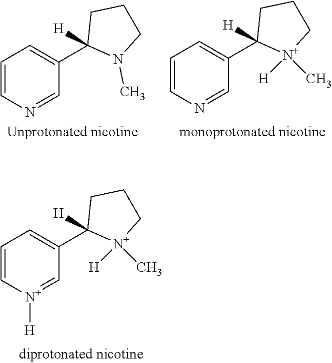

Reference in the specification to protonated form means both monoprotonated nicotine and diprotonated nicotine. Reference in the specification to amounts in the protonated form means the combined amount of monoprotonated nicotine and diprotonated nicotine. Furthermore, when reference is made to a fully protonated formulation it will be understood that at any one time there may be very minor amounts of unprotonated nicotine present, e.g. less than 1% unprotonated.

For ease of reference, these and further aspects of the present disclosure are now discussed under appropriate section headings. However, the teachings under each section are not necessarily limited to each particular section.

Energy of Binding

As discussed herein, in the presence of water the energy of binding of the one or more encapsulating materials with the one or more flavors to be encapsulated is from −0.5 to −8 kcal/mol.

In one aspect the energy of binding may be determined by molecular modelling and, in particular, using docking performed in Autodock 4.2 [The Scripps Research Institute, La Jolla, Calif., USA] (Morris, G. M., Huey, R., Lindstrom, W., Sanner, M. F., Belew, R. K., Goodsell, D. S., & Olson, A. J. (2009). Autodock4 and AutodockTools4: automated docking with selective receptor flexibility. J. Computational Chemistry, 2785-2791). The docking performed in Autodock 4.2 may be performed using the following settings:

Grid Point Spacing (Angstroms): 0.375

Number of grid points in each Cartesian direction
x: 40
y: 40
z: 40

User-specified initial position for ligand: random

Initial relative dihedral offset: random

User-specified initial relative dihedrals: random

Docking search parameter: Genetic Algorithm

Number of requested GA dockings: 10 runs

Population size: 150

Maximum number of evaluations: 2500000

Maximum number of top individuals that automatically survive: 1

Rate of gene mutation: 0.02

Rate of crossover: 0.08

GA crossover mode: “twopt”

Mean of Cauchy distribution for gene mutation(alpha parameter): 0

Variance of Cauchy distribution for gene mutation(beta parameter): 1

Number of generations fro picking worst individuals: 10

Docking output: Lamarckian Ga.

In one aspect, in the presence of water the energy of binding of the one or more encapsulating materials with the one or more flavors to be encapsulated is from −1 to −8 kcal/mol. In one aspect, in the presence of water the energy of binding of the one or more encapsulating materials with the one or more flavors to be encapsulated is from −1.5 to −8 kcal/mol. In one aspect, in the presence of water the energy of binding of the one or more encapsulating materials with the one or more flavors to be encapsulated is from −2 to −8 kcal/mol. In one aspect, in the presence of water the energy of binding of the one or more encapsulating materials with the one or more flavors to be encapsulated is from −2.5 to −8 kcal/mol. In one aspect, in the presence of water the energy of binding of the one or more encapsulating materials with the one or more flavors to be encapsulated is from −3 to −8 kcal/mol. In one aspect, in the presence of water the energy of binding of the one or more encapsulating materials with the one or more flavors to be encapsulated is from −3.5 to −8 kcal/mol. In one aspect, in the presence of water the energy of binding of the one or more encapsulating materials with the one or more flavors to be encapsulated is from −4 to −8 kcal/mol. In one aspect, in the presence of water the energy of binding of the one or more encapsulating materials with the one or more flavors to be encapsulated is from −4.5 to −8 kcal/mol. In one aspect, in the presence of water the energy of binding of the one or more encapsulating materials with the one or more flavors to be encapsulated is from −5 to −8 kcal/mol. In one aspect, in the presence of water the energy of binding of the one or more encapsulating materials with the one or more flavors to be encapsulated is from −5 to −7.5 kcal/mol. In one aspect, in the presence of water the energy of binding of the one or more encapsulating materials with the one or more flavors to be encapsulated is from −5 to −7 kcal/mol.

In one aspect, in the presence of water the energy of binding of the one or more encapsulating materials with the one or more flavors to be encapsulated is from −0.5 to −7.5 kcal/mol. In one aspect, in the presence of water the energy of binding of the one or more encapsulating materials with the one or more flavors to be encapsulated is from −0.5 to −7 kcal/mol. In one aspect, in the presence of water the energy of binding of the one or more encapsulating materials with the one or more flavors to be encapsulated is from −0.5 to −6.5 kcal/mol. In one aspect, in the presence of water the energy of binding of the one or more encapsulating materials with the one or more flavors to be encapsulated is from −0.5 to −6 kcal/mol. In one aspect, in the presence of water the energy of binding of the one or more encapsulating materials with the one or more flavors to be encapsulated is from −1 to −6 kcal/mol. In one aspect, in the presence of water the energy of binding of the one or more encapsulating materials with the one or more flavors to be encapsulated is from −1.5 to −6 kcal/mol. In one aspect, in the presence of water the energy of binding of the one or more encapsulating materials with the one or more flavors to be encapsulated is from −2 to −6 kcal/mol. In one aspect, in the presence of water the energy of binding of the one or more encapsulating materials with the one or more flavors to be encapsulated is from −2.5 to −6 kcal/mol. In one aspect, in the presence of water the energy of binding of the one or more encapsulating materials with the one or more flavors to be encapsulated is from −3 to −6 kcal/mol. In one aspect, in the presence of water the energy of binding of the one or more encapsulating materials with the one or more flavors to be encapsulated is from −3.5 to −6 kcal/mol. In one aspect, in the presence of water the energy of binding of the one or more encapsulating materials with the one or more flavors to be encapsulated is from −4 to −6 kcal/mol. In one aspect, in the presence of water the energy of binding of the one or more encapsulating materials with the one or more flavors to be encapsulated is from −4.5 to −6 kcal/mol. In one aspect, in the presence of water the energy of binding of the one or more encapsulating materials with the one or more flavors to be encapsulated is from −5 to −6 kcal/mol.

Water

As discussed herein the aerosolizable formulation contains water. In one aspect water is present in an amount of at least 30 wt. % based on the aerosolizable formulation. In one aspect water is present in an amount of at least 35 wt. % based on the aerosolizable formulation. In one aspect water is present in an amount of at least 40 wt. % based on the aerosolizable formulation. In one aspect water is present in an amount of at least 45 wt. % based on the aerosolizable formulation. In one aspect water is present in an amount of at least 50 wt. % based on the aerosolizable formulation. In one aspect water is present in an amount of at least 55 wt. % based on the aerosolizable formulation. In one aspect water is present in an amount of at least 60 wt. % based on the aerosolizable formulation. In one aspect water is present in an amount of at least 65 wt. % based on the aerosolizable formulation. In one aspect water is present in an amount of at least 70 wt. % based on the aerosolizable formulation. In one aspect water is present in an amount of at least 75 wt. % based on the aerosolizable formulation. In one aspect water is present in an amount of at least 80 wt. % based on the aerosolizable formulation. In one aspect water is present in an amount of at least 85 wt. % based on the aerosolizable formulation. In one aspect water is present in an amount of at least 90 wt. % based on the aerosolizable formulation. In one aspect water is present in an amount of at least 95 wt. % based on the aerosolizable formulation. In one aspect water is present in an amount of at least 99 wt. % based on the aerosolizable formulation.

In one aspect water is present in an amount of from 30 to 99 wt. % based on the aerosolizable formulation. In one aspect water is present in an amount of from 35 to 99 wt. % based on the aerosolizable formulation. In one aspect water is present in an amount of from 40 to 99 wt. % based on the aerosolizable formulation. In one aspect water is present in an amount of from 45 to 99 wt. % based on the aerosolizable formulation. In one aspect water is present in an amount of from 50 to 99 wt. % based on the aerosolizable formulation. In one aspect water is present in an amount of from 55 to 99 wt. % based on the aerosolizable formulation. In one aspect water is present in an amount of from 60 to 99 wt. % based on the aerosolizable formulation. In one aspect water is present in an amount of from 65 to 99 wt. % based on the aerosolizable formulation. In one aspect water is present in an amount of from 70 to 99 wt. % based on the aerosolizable formulation. In one aspect water is present in an amount of from 75 to 99 wt. % based on the aerosolizable formulation. In one aspect water is present in an amount of from 80 to 99 wt. % based on the aerosolizable formulation. In one aspect water is present in an amount of from 85 to 99 wt. % based on the aerosolizable formulation. In one aspect water is present in an amount of from 90 to 99 wt. % based on the aerosolizable formulation. In one aspect water is present in an amount of from 95 to 99 wt. % based on the aerosolizable formulation.

The use of water allows for the replacement of some or all of the glycerol, propylene glycol, 1,3-propane diol and mixtures thereof typically used in e-cigarettes. In one aspect the aerosolizable formulation contains glycerol, propylene glycol, 1,3-propane diol and mixtures thereof in a combined amount of no greater than 10 wt. % based on the aerosolizable formulation. In one aspect the aerosolizable formulation contains glycerol, propylene glycol, 1,3-propane diol and mixtures thereof in a combined amount of no greater than 8 wt. % based on the aerosolizable formulation. In one aspect the aerosolizable formulation contains glycerol, propylene glycol, 1,3-propane diol and mixtures thereof in a combined amount of no greater than 5 wt. % based on the aerosolizable formulation. In one aspect the aerosolizable formulation contains glycerol, propylene glycol, 1,3-propane diol and mixtures thereof in a combined amount of no greater than 2 wt. % based on the aerosolizable formulation. In one aspect the aerosolizable formulation contains glycerol, propylene glycol, 1,3-propane diol and mixtures thereof in a combined amount of no greater than 1 wt. % based on the aerosolizable formulation. In one aspect the aerosolizable formulation contains glycerol, propylene glycol, 1,3-propane diol and mixtures thereof in a combined amount of no greater than 0.5 wt. % based on the aerosolizable formulation. In one aspect the aerosolizable formulation contains glycerol, propylene glycol, 1,3-propane diol and mixtures thereof in a combined amount of no greater than 0.2 wt. % based on the aerosolizable formulation. In one aspect the aerosolizable formulation contains glycerol, propylene glycol, 1,3-propane diol and mixtures thereof in a combined amount of no greater than 0.1 wt. % based on the aerosolizable formulation. In one aspect the aerosolizable formulation contains glycerol, propylene glycol, 1,3-propane diol and mixtures thereof in a combined amount of no greater than 0.01 wt. % based on the aerosolizable formulation. In one aspect the aerosolizable formulation contains no glycerol, propylene glycol, 1,3-propane diol and mixtures thereof.

In one aspect the aerosolizable formulation contains glycerol, propylene glycol, and mixtures thereof in a combined amount of no greater than 10 wt. % based on the aerosolizable formulation. In one aspect the aerosolizable formulation contains glycerol, propylene glycol, and mixtures thereof in a combined amount of no greater than 8 wt. % based on the aerosolizable formulation. In one aspect the aerosolizable formulation contains glycerol, propylene glycol, and mixtures thereof in a combined amount of no greater than 5 wt. % based on the aerosolizable formulation. In one aspect the aerosolizable formulation contains glycerol, propylene glycol, and mixtures thereof in a combined amount of no greater than 2 wt. % based on the aerosolizable formulation. In one aspect the aerosolizable formulation contains glycerol, propylene glycol, and mixtures thereof in a combined amount of no greater than 1 wt. % based on the aerosolizable formulation. In one aspect the aerosolizable formulation contains glycerol, propylene glycol, and mixtures thereof in a combined amount of no greater than 0.5 wt. % based on the aerosolizable formulation. In one aspect the aerosolizable formulation contains glycerol, propylene glycol, and mixtures thereof in a combined amount of no greater than 0.2 wt. % based on the aerosolizable formulation. In one aspect the aerosolizable formulation contains glycerol, propylene glycol, and mixtures thereof in a combined amount of no greater than 0.1 wt. % based on the aerosolizable formulation. In one aspect the aerosolizable formulation contains glycerol, propylene glycol, and mixtures thereof in a combined amount of no greater than 0.01 wt. % based on the aerosolizable formulation. In one aspect the aerosolizable formulation contains no glycerol, propylene glycol, and mixtures thereof.

In one aspect the aerosolizable formulation contains glycerol in an amount of no greater than 10 wt. % based on the aerosolizable formulation. In one aspect the aerosolizable formulation contains glycerol in an amount of no greater than 8 wt. % based on the aerosolizable formulation. In one aspect the aerosolizable formulation contains glycerol in an amount of no greater than 5 wt. % based on the aerosolizable formulation. In one aspect the aerosolizable formulation contains glycerol in an amount of no greater than 2 wt. % based on the aerosolizable formulation. In one aspect the aerosolizable formulation contains glycerol in an amount of no greater than 1 wt. % based on the aerosolizable formulation. In one aspect the aerosolizable formulation contains glycerol in an amount of no greater than 0.5 wt. % based on the aerosolizable formulation. In one aspect the aerosolizable formulation contains glycerol in an amount of no greater than 0.2 wt. % based on the aerosolizable formulation. In one aspect the aerosolizable formulation contains glycerol in an amount of no greater than 0.1 wt. % based on the aerosolizable formulation. In one aspect the aerosolizable formulation contains glycerol in an amount of no greater than 0.01 wt. % based on the aerosolizable formulation. In one aspect the aerosolizable formulation contains no glycerol.

In one aspect the aerosolizable formulation contains propylene glycol in an amount of no greater than 10 wt. % based on the aerosolizable formulation. In one aspect the aerosolizable formulation contains propylene glycol in an amount of no greater than 8 wt. % based on the aerosolizable formulation. In one aspect the aerosolizable formulation contains propylene glycol in an amount of no greater than 5 wt. % based on the aerosolizable formulation. In one aspect the aerosolizable formulation contains propylene glycol in an amount of no greater than 2 wt. % based on the aerosolizable formulation. In one aspect the aerosolizable formulation contains propylene glycol in an amount of no greater than 1 wt. % based on the aerosolizable formulation. In one aspect the aerosolizable formulation contains propylene glycol in an amount of no greater than 0.5 wt. % based on the aerosolizable formulation. In one aspect the aerosolizable formulation contains propylene glycol in an amount of no greater than 0.2 wt. % based on the aerosolizable formulation. In one aspect the aerosolizable formulation contains propylene glycol in an amount of no greater than 0.1 wt. % based on the aerosolizable formulation. In one aspect the aerosolizable formulation contains propylene glycol in an amount of no greater than 0.01 wt. % based on the aerosolizable formulation. In one aspect the aerosolizable formulation contains no propylene glycol.

Nicotine

Nicotine formulations may be provided having desirable properties of flavor, impact, irritation, smoothness and/or nicotine reward for the user. In one aspect nicotine is present in an amount of no greater than 6 wt % based on the total weight of the aerosolizable formulation. In one aspect nicotine is present in an amount of from 0.01 to 6 wt % based on the total weight of the aerosolizable formulation. In one aspect nicotine is present in an amount of from 0.02 to 6 wt % based on the total weight of the aerosolizable formulation. In one aspect nicotine is present in an amount of from 0.05 to 6 wt % based on the total weight of the aerosolizable formulation. In one aspect nicotine is present in an amount of from 0.08 to 6 wt % based on the total weight of the aerosolizable formulation. In one aspect nicotine is present in an amount of from 0.01 to 5 wt % based on the total weight of the aerosolizable formulation. In one aspect nicotine is present in an amount of from 0.02 to 5 wt % based on the total weight of the aerosolizable formulation. In one aspect nicotine is present in an amount of from 0.05 to 5 wt % based on the total weight of the aerosolizable formulation. In one aspect nicotine is present in an amount of from 0.08 to 5 wt % based on the total weight of the aerosolizable formulation. In one aspect nicotine is present in an amount of no greater than 4 wt % based on the total weight of the aerosolizable formulation. In one aspect nicotine is present in an amount of from 0.01 to 4 wt % based on the total weight of the aerosolizable formulation. In one aspect nicotine is present in an amount of from 0.02 to 4 wt % based on the total weight of the aerosolizable formulation. In one aspect nicotine is present in an amount of from 0.05 to 4 wt % based on the total weight of the aerosolizable formulation. In one aspect nicotine is present in an amount of from 0.08 to 4 wt % based on the total weight of the aerosolizable formulation. In one aspect nicotine is present in an amount of no greater than 3 wt % based on the total weight of the aerosolizable formulation. In one aspect nicotine is present in an amount of from 0.01 to 3 wt % based on the total weight of the aerosolizable formulation. In one aspect nicotine is present in an amount of from 0.02 to 3 wt % based on the total weight of the aerosolizable formulation. In one aspect nicotine is present in an amount of from 0.05 to 3 wt % based on the total weight of the aerosolizable formulation. In one aspect nicotine is present in an amount of from 0.08 to 3 wt % based on the total weight of the aerosolizable formulation. In one aspect nicotine is present in an amount of no greater than 2 wt % based on the total weight of the aerosolizable formulation. In one aspect nicotine is present in an amount of from 0.01 to 2 wt % based on the total weight of the aerosolizable formulation. In one aspect nicotine is present in an amount of from 0.02 to 2 wt % based on the total weight of the aerosolizable formulation. In one aspect nicotine is present in an amount of from 0.05 to 2 wt % based on the total weight of the aerosolizable formulation. In one aspect nicotine is present in an amount of from 0.08 to 2 wt % based on the total weight of the aerosolizable formulation. In one aspect nicotine is present in an amount of no greater than 1 wt % based on the total weight of the aerosolizable formulation. In one aspect nicotine is present in an amount of from 0.01 to 1 wt % based on the total weight of the aerosolizable formulation. In one aspect nicotine is present in an amount of from 0.02 to 1 wt % based on the total weight of the aerosolizable formulation. In one aspect nicotine is present in an amount of from 0.05 to 1 wt % based on the total weight of the aerosolizable formulation. In one aspect nicotine is present in an amount of from 0.08 to 1 wt % based on the total weight of the aerosolizable formulation. In one aspect nicotine is present in an amount of from 0.1 to 1 wt % based on the total weight of the aerosolizable formulation. In one aspect nicotine is present in an amount of no greater than 0.6 wt % based on the total weight of the aerosolizable formulation. In one aspect nicotine is present in an amount of from 0.01 to 0.6 wt % based on the total weight of the aerosolizable formulation. In one aspect nicotine is present in an amount of from 0.02 to 0.6 wt % based on the total weight of the aerosolizable formulation. In one aspect nicotine is present in an amount of from 0.05 to 0.6 wt % based on the total weight of the aerosolizable formulation. In one aspect nicotine is present in an amount of from 0.08 to 0.6 wt % based on the total weight of the aerosolizable formulation. In one aspect nicotine is present in an amount of from 0.1 to 0.6 wt % based on the total weight of the aerosolizable formulation. In one aspect nicotine is present in an amount of no greater than 0.5 wt % based on the total weight of the aerosolizable formulation. In one aspect nicotine is present in an amount of from 0.01 to 0.5 wt % based on the total weight of the aerosolizable formulation. In one aspect nicotine is present in an amount of from 0.02 to 0.5 wt % based on the total weight of the aerosolizable formulation. In one aspect nicotine is present in an amount of from 0.05 to 0.5 wt % based on the total weight of the aerosolizable formulation. In one aspect nicotine is present in an amount of from 0.08 to 0.5 wt % based on the total weight of the aerosolizable formulation. In one aspect nicotine is present in an amount of no greater than 0.2 wt % based on the total weight of the aerosolizable formulation. In one aspect nicotine is present in an amount of from 0.01 to 0.2 wt % based on the total weight of the aerosolizable formulation. In one aspect nicotine is present in an amount of from 0.02 to 0.2 wt % based on the total weight of the aerosolizable formulation. In one aspect nicotine is present in an amount of from 0.05 to 0.2 wt % based on the total weight of the aerosolizable formulation. In one aspect nicotine is present in an amount of from 0.08 to 0.2 wt % based on the total weight of the aerosolizable formulation. In one aspect nicotine is present in an amount of no greater than 0.1 wt % based on the total weight of the aerosolizable formulation. In one aspect nicotine is present in an amount of from 0.01 to 0.1 wt % based on the total weight of the aerosolizable formulation. In one aspect nicotine is present in an amount of from 0.02 to 0.1 wt % based on the total weight of the aerosolizable formulation. In one aspect nicotine is present in an amount of from 0.05 to 0.1 wt % based on the total weight of the aerosolizable formulation. In one aspect nicotine is present in an amount of from 0.08 to 0.1 wt % based on the total weight of the aerosolizable formulation.

The formulation may comprise nicotine in protonated form. The formulation may comprise nicotine in unprotonated form. In one aspect the formulation comprises nicotine in unprotonated form and nicotine in monoprotonated form. In one aspect the formulation comprises nicotine in unprotonated form and nicotine in diprotonated form. In one aspect the formulation comprises nicotine in unprotonated form, nicotine in monoprotonated form and nicotine in diprotonated form.

In one aspect at least 5 wt % of the nicotine present in the formulation is in protonated form. In one aspect at least 10 wt % of the nicotine present in the formulation is in protonated form. In one aspect at least 15 wt % of the nicotine present in the formulation is in protonated form. In one aspect at least 20 wt % of the nicotine present in the formulation is in protonated form. In one aspect at least 25 wt % of the nicotine present in the formulation is in protonated form. In one aspect at least 30 wt % of the nicotine present in the formulation is in protonated form. In one aspect at least 35 wt % of the nicotine present in the formulation is in protonated form. In one aspect at least 40 wt % of the nicotine present in the formulation is in protonated form. In one aspect at least 45 wt % of the nicotine present in the formulation is in protonated form. In one aspect at least 50 wt % of the nicotine present in the formulation is in protonated form. In one aspect at least 55 wt % of the nicotine present in the formulation is in protonated form. In one aspect at least 60 wt % of the nicotine present in the formulation is in protonated form. In one aspect at least 65 wt % of the nicotine present in the formulation is in protonated form. In one aspect at least 70 wt % of the nicotine present in the formulation is in protonated form. In one aspect at least 75 wt % of the nicotine present in the formulation is in protonated form. In one aspect at least 80 wt % of the nicotine present in the formulation is in protonated form. In one aspect at least 85 wt % of the nicotine present in the formulation is in protonated form. In one aspect at least 90 wt % of the nicotine present in the formulation is in protonated form. In one aspect at least 95 wt % of the nicotine present in the formulation is in protonated form. In one aspect at least 99 wt % of the nicotine present in the formulation is in protonated form. In one aspect at least 99.9 wt % of the nicotine present in the formulation is in protonated form.

In one aspect from 50 to 95 wt % of the nicotine present in the formulation is in protonated form. In one aspect from 55 to 95 wt % of the nicotine present in the formulation is in protonated form. In one aspect from 60 to 95 wt % of the nicotine present in the formulation is in protonated form. In one aspect from 65 to 95 wt % of the nicotine present in the formulation is in protonated form. In one aspect from 70 to 95 wt % of the nicotine present in the formulation is in protonated form. In one aspect from 75 to 95 wt % of the nicotine present in the formulation is in protonated form. In one aspect from 80 to 95 wt % of the nicotine present in the formulation is in protonated form. In one aspect from 85 to 95 wt % of the nicotine present in the formulation is in protonated form. In one aspect from 90 to 95 wt % of the nicotine present in the formulation is in protonated form.

In one aspect from 50 to 99 wt % of the nicotine present in the formulation is in protonated form. In one aspect from 55 to 99 wt % of the nicotine present in the formulation is in protonated form. In one aspect from 60 to 99 wt % of the nicotine present in the formulation is in protonated form. In one aspect from 65 to 99 wt % of the nicotine present in the formulation is in protonated form. In one aspect from 70 to 99 wt % of the nicotine present in the formulation is in protonated form. In one aspect from 75 to 99 wt % of the nicotine present in the formulation is in protonated form. In one aspect from 80 to 99 wt % of the nicotine present in the formulation is in protonated form. In one aspect from 85 to 99 wt % of the nicotine present in the formulation is in protonated form. In one aspect from 90 to 99 wt % of the nicotine present in the formulation is in protonated form.

The relevant amounts of nicotine which are present in the formulation in protonated form are specified herein. These amounts may be readily calculated by one skilled in the art. Nicotine, 3-(1-methylpyrrolidin-2-yl) pyridine, is a diprotic base with pKa of 3.12 for the pyridine ring and 8.02 for the pyrrolidine ring It can exist in pH-dependent protonated (mono- and di-) and non-protonated (free base) forms which have different bioavailability.

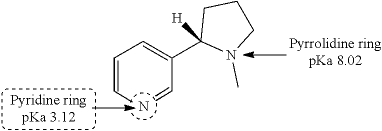

The distribution of protonated and non-protonated nicotine will vary at various pH increments.

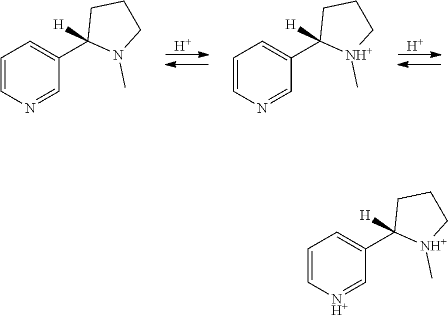

The fraction of non-protonated nicotine will be predominant at high pH levels whilst a decrease in the pH will see an increase of the fraction of protonated nicotine (mono- or di-depending on the pH). If the relative fraction of protonated nicotine and the total amount of nicotine in the sample are known, the absolute amount of protonated nicotine can be calculated.

The relative fraction of protonated nicotine in formulation can be calculated by using the Henderson-Hasselbalch equation, which describes the pH as a derivation of the acid dissociation constant equation, and it is extensively employed in chemical and biological systems. Consider the following equilibrium:

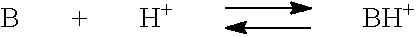

The Henderson-Hasselbalch equation for this equilibrium is:

$$pH = pK a + log \frac{[ B ]}{[ BH + ]}$$

Where [B] is the amount of non-protonated nicotine (i.e. free base), [BH+] the amount of protonated nicotine (i.e. conjugate acid) and pKa is the reference pKa value for the pyrrolidine ring nitrogen of nicotine (pKa=8.02). The relative fraction of protonated nicotine can be derived from the alpha value of the non-protonated nicotine calculated from the Henderson-Hasselbalch equation as:

$$\% protonated nicotine = 100 - \{ \frac{\frac{[ B ]}{[ BH + ]}}{\{ 1 + \frac{[ B ]}{[ B H + ]} \}} * 100 \}$$

Determination of pKa values of nicotine formulations was carried out using the basic approach described in “Spectroscopic investigations into the acid-base properties of nicotine at different temperatures”, Peter M. Clayton, Carl A. Vas, Tam T. T. Bui, Alex F. Drake and Kevin McAdam, Anal. Methods, 2013, 5, 81-88.

Acid

In one aspect the aerosolizable formulation further comprises an acid. The acid may be any suitable acid. In one aspect the acid is an organic acid. In one aspect the acid is a carboxylic acid. In one aspect the acid is an organic carboxylic acid.

In one aspect the acid is selected from the group consisting of acetic acid, lactic acid, formic acid, citric acid, benzoic acid, pyruvic acid, levulinic acid, succinic acid, tartaric acid, sorbic acid, propionic acid, phenylacetic acid, and mixtures thereof. In one aspect the acid is selected from the group consisting of citric acid, benzoic acid, levulinic acid, lactic acid, sorbic acid, and mixtures thereof. In one aspect the acid is selected from the group consisting of citric acid, benzoic acid, levulinic acid, and mixtures thereof. In one aspect the acid is at least citric acid. In one aspect the acid consists of citric acid.

In one aspect the acid is selected from acids having a pKa[[pka]] of from 2 to 5. In one aspect the acid is a weak acid. In one aspect the acid is a weak organic acid.

In one aspect the acid has a solubility in water of at least 2 g/L at 20° C. In one aspect the acid has a solubility in water of at least 5 g/L at 20° C. In one aspect the acid has a solubility in water of at least 10 g/L at 20° C. In one aspect the acid has a solubility in water of at least 20 g/L at 20° C. In one aspect the acid has a solubility in water of at least 50 g/L at 20° C. In one aspect the acid has a solubility in water of at least 100 g/L at 20° C. In one aspect the acid has a solubility in water of at least 200 g/L at 20° C. In one aspect the acid has a solubility in water of at least 300 g/L at 20° C. In one aspect the acid has a solubility in water of at least 400 g/L at 20° C. In one aspect the acid has a solubility in water of at least 500 g/L at 20° C. In one aspect the acid has a solubility in water of at least 600 g/L at 20° C. In one aspect the acid has a solubility in water of at least 700 g/L at 20° C. In one aspect the acid has a solubility in water of at least 800 g/L at 20° C. In one aspect the acid has a solubility in water of at least 900 g/L at 20° C. In one aspect the acid has a solubility in water of at least 1000 g/L at 20° C. In one aspect the acid has a solubility in water of at least 1100 g/L at 20° C.

The molar ratio of acid to nicotine may be selected as desired. In one aspect the molar ratio of acid to nicotine is from 5:1 to 1:5. In one aspect the molar ratio of acid to nicotine is from 4:1 to 1:4. In one aspect the molar ratio of acid to nicotine is from 3:1 to 1:3. In one aspect the molar ratio of acid to nicotine is from 2:1 to 1:2. In one aspect the molar ratio of acid to nicotine is from 1.5:1 to 1:1.5. In one aspect the molar ratio of acid to nicotine is from 1.2:1 to 1:1.2. In one aspect the molar ratio of acid to nicotine is from 5:1 to 1:1. In one aspect the molar ratio of acid to nicotine is from 4:1 to 1:1. In one aspect the molar ratio of acid to nicotine is from 3:1 to 1:1. In one aspect the molar ratio of acid to nicotine is from 2:1 to 1:1. In one aspect the molar ratio of acid to nicotine is from 1.5:1 to 1:1. In one aspect the molar ratio of acid to nicotine is from 1.2:1 to 1:1.

In one aspect the total content of acid present in the formulation is no greater than 5 mole equivalents based on the nicotine. In one aspect the total content of acid present in the formulation is no greater than 4 mole equivalents based on the nicotine. In one aspect the total content of acid present in the formulation is no greater than 3 mole equivalents based on the nicotine. In one aspect the total content of acid present in the formulation is no greater than 2 mole equivalents based on the nicotine. In one aspect the total content of acid present in the formulation is no greater than 1 mole equivalents based on the nicotine.

In one aspect the total content of acid present in the formulation is no less than 0.01 mole equivalents based on the nicotine. In one aspect the total content of acid present in the formulation is no less than 0.05 mole equivalents based on the nicotine. In one aspect the total content of acid present in the formulation is no less than 0.1 mole equivalents based on the nicotine. In one aspect the total content of acid present in the formulation is no less than 0.2 mole equivalents based on the nicotine. In one aspect the total content of acid present in the formulation is no less than 0.3 mole equivalents based on the nicotine. In one aspect the total content of acid present in the formulation is no less than 0.4 mole equivalents based on the nicotine. In one aspect the total content of acid present in the formulation is no less than 0.5 mole equivalents based on the nicotine. In one aspect the total content of acid present in the formulation is no less than 0.7 mole equivalents based on the nicotine.

The acid may be present in any suitable amount. In one aspect the acid is present in an amount of no greater than 6 wt % based on the aerosolizable formulation. In one aspect the acid is present in an amount of from 0.01 to 6 wt % based on the aerosolizable formulation. In one aspect the acid is present in an amount of from 0.02 to 6 wt % based on the aerosolizable formulation. In one aspect the acid is present in an amount of from 0.05 to 6 wt % based on the aerosolizable formulation. In one aspect the acid is present in an amount of from 0.08 to 6 wt % based on the aerosolizable formulation. In one aspect the acid is present in an amount of from 0.01 to 5 wt % based on the aerosolizable formulation. In one aspect the acid is present in an amount of from 0.02 to 5 wt % based on the aerosolizable formulation. In one aspect the acid is present in an amount of from 0.05 to 5 wt % based on the aerosolizable formulation. In one aspect the acid is present in an amount of from 0.08 to 5 wt % based on the aerosolizable formulation. In one aspect the acid is present in an amount of no greater than 4 wt % based on the aerosolizable formulation. In one aspect the acid is present in an amount of from 0.01 to 4 wt % based on the aerosolizable formulation. In one aspect the acid is present in an amount of from 0.02 to 4 wt % based on the aerosolizable formulation. In one aspect the acid is present in an amount of from 0.05 to 4 wt % based on the aerosolizable formulation. In one aspect the acid is present in an amount of from 0.08 to 4 wt % based on the aerosolizable formulation. In one aspect the acid is present in an amount of no greater than 3 wt % based on the aerosolizable formulation. In one aspect the acid is present in an amount of from 0.01 to 3 wt % based on the aerosolizable formulation. In one aspect the acid is present in an amount of from 0.02 to 3 wt % based on the aerosolizable formulation. In one aspect the acid is present in an amount of from 0.05 to 3 wt % based on the aerosolizable formulation. In one aspect the acid is present in an amount of from 0.08 to 3 wt % based on the aerosolizable formulation. In one aspect the acid is present in an amount of no greater than 2 wt % based on the aerosolizable formulation. In one aspect the acid is present in an amount of from 0.01 to 2 wt % based on the aerosolizable formulation. In one aspect the acid is present in an amount of from 0.02 to 2 wt % based on the aerosolizable formulation. In one aspect the acid is present in an amount of from 0.05 to 2 wt % based on the aerosolizable formulation. In one aspect the acid is present in an amount of from 0.08 to 2 wt % based on the aerosolizable formulation. In one aspect the acid is present in an amount of no greater than 1 wt % based on the aerosolizable formulation. In one aspect the acid is present in an amount of from 0.01 to 1 wt % based on the aerosolizable formulation. In one aspect the acid is present in an amount of from 0.02 to 1 wt % based on the aerosolizable formulation. In one aspect the acid is present in an amount of from 0.05 to 1 wt % based on the aerosolizable formulation. In one aspect the acid is present in an amount of from 0.08 to 1 wt % based on the aerosolizable formulation. In one aspect the acid is present in an amount of from 0.1 to 1 wt % based on the aerosolizable formulation. In one aspect the acid is present in an amount of no greater than 0.6 wt % based on the aerosolizable formulation. In one aspect the acid is present in an amount of from 0.01 to 0.6 wt % based on the aerosolizable formulation. In one aspect the acid is present in an amount of from 0.02 to 0.6 wt % based on the aerosolizable formulation. In one aspect the acid is present in an amount of from 0.05 to 0.6 wt % based on the aerosolizable formulation. In one aspect the acid is present in an amount of from 0.08 to 0.6 wt % based on the aerosolizable formulation. In one aspect the acid is present in an amount of from 0.1 to 0.6 wt % based on the aerosolizable formulation. In one aspect the acid is present in an amount of no greater than 0.5 wt % based on the aerosolizable formulation. In one aspect the acid is present in an amount of from 0.01 to 0.5 wt % based on the aerosolizable formulation. In one aspect the acid is present in an amount of from 0.02 to 0.5 wt % based on the aerosolizable formulation. In one aspect the acid is present in an amount of from 0.05 to 0.5 wt % based on the aerosolizable formulation. In one aspect the acid is present in an amount of from 0.08 to 0.5 wt % based on the aerosolizable formulation. In one aspect the acid is present in an amount of no greater than 0.2 wt % based on the aerosolizable formulation. In one aspect the acid is present in an amount of from 0.01 to 0.2 wt % based on the aerosolizable formulation. In one aspect the acid is present in an amount of from 0.02 to 0.2 wt % based on the aerosolizable formulation. In one aspect the acid is present in an amount of from 0.05 to 0.2 wt % based on the aerosolizable formulation. In one aspect the acid is present in an amount of from 0.08 to 0.2 wt % based on the aerosolizable formulation. In one aspect the acid is present in an amount of no greater than 0.1 wt % based on the aerosolizable formulation. In one aspect the acid is present in an amount of from 0.01 to 0.1 wt % based on the aerosolizable formulation. In one aspect the acid is present in an amount of from 0.02 to 0.1 wt % based on the aerosolizable formulation. In one aspect the acid is present in an amount of from 0.05 to 0.1 wt % based on the aerosolizable formulation. In one aspect the acid is present in an amount of from 0.08 to 0.1 wt % based on the aerosolizable formulation.

The amount of acid and the solubility of the acid may be selected such that a given amount of the acid will dissolve in the water. In one aspect at 20° C. at least 20% of the acid dissolves in the water. In one aspect at 25° C. at least 20% of the acid dissolves in the water. In one aspect at 30° C. at least 20% of the acid dissolves in the water. In one aspect at 20° C. at least 35% of the acid dissolves in the water. In one aspect at 20° C. at least 40% of the acid dissolves in the water. In one aspect at 20° C. at least 45% of the acid dissolves in the water. In one aspect at 20° C. at least 50% of the acid dissolves in the water. In one aspect at 20° C. at least 55% of the acid dissolves in the water.

Flavor

The aerosolizable formulation comprises one or more flavors or flavoring components. As used herein, the terms “flavor” and “flavorant” refer to materials which, where local regulations permit, may be used to create a desired taste or aroma in a product for adult consumers. They may include extracts (e.g. liquorice, hydrangea, Japanese white bark magnolia leaf, chamomile, fenugreek, clove, menthol, Japanese mint, aniseed, cinnamon, herb, wintergreen, cherry, berry, peach, apple, Drambuie, bourbon, scotch, whiskey, spearmint, peppermint, lavender, cardamom, celery, cascarilla, nutmeg, sandalwood, bergamot, geranium, honey essence, rose oil, vanilla, lemon oil, orange oil, cassia, caraway, cognac, jasmine, ylang-ylang, sage, fennel, piment, ginger, anise, coriander, coffee, or a mint oil from any species of the genus Mentha), flavor enhancers, bitterness receptor site blockers, sensorial receptor site activators or stimulators, sugars and/or sugar substitutes (e.g., sucralose, acesulfame potassium, aspartame, saccharine, cyclamates, lactose, sucrose, glucose, fructose, sorbitol, or mannitol), and other additives such as charcoal, chlorophyll, minerals, botanicals, or breath freshening agents. They may be imitation, synthetic or natural ingredients or blends thereof. They may be in any suitable form, for example, oil, liquid, or powder.

The one or more flavors may be selected from dodecan-1-ol; octan-1-ol; 4-methyl-1-propan-2-yl-7-oxabicyclo[2.2.1]heptane; 2,4,5-trimethylphenol; 2,4,6-trimethylphenol; 2,4-dimethylbenzaldehyde; 2-ethoxy-3-methylpyrazine; 2-ethylphenol; 2-ethylpyrazine; 2-methoxy-3-(2-methylpropyl)pyrazine; 5-methyl-2-propan-2-ylhex-2-enal; 2-methylpent-2-enoic acid; 2-butan-2-yl-3-methoxypyrazine; 3,5,5-trimethylcyclohexane-1,2-dione; 3-ethylphenol; [(z)-hex-3-enyl] 3-methylbutanoate; 3-methylnonane-2,4-dione; 3-methylbut-2-ene-1-thiol; 3-methyl-1h-indole; 3-methylphenol; 3-propylphenol; oxolan-2-one; 4-(hydroxymethyl)-2-methoxyphenol; 4-methylphenol; 2-methoxy-4-propylphenol; 4-propylphenol; [4-(3-oxobutyl)phenyl] acetate; 5-ethyl-2-methoxyphenol; (e)-5-methylhept-2-en-4-one; (e)-5-methyl-2-phenylhex-2-enal; 5-vinyl-2,3-dimethylpyrazine; 2-ethyl-6-methoxyphenol; 2-methoxy-6-methylphenol; 6-methylchromen-2-one; 1,1-diethoxyethane; 1-(4-methoxyphenyl)ethanone; acetic acid; 3-hydroxybutan-2-one; 1-phenylethanone; hexane-2,3-dione; 1-pyrazin-2-ylethanone; 1-(2-pyridyl)ethanone; 1-pyridin-4-ylethanone; thiazol-2-yl)ethanone; 1-(5-methylfuran-2-yl)ethanone; prop-2-enyl 6-cyclohexylhexanoate; prop-2-enyl hexanoate; prop-2-enyl nonanoate; pentyl butanoate; (2z)-2-(phenylmethylidene)heptanal; pentyl hexanoate; 1-methoxy-4-[(e)-prop-1-enyl] benzene; (4-methoxyphenyl)methyl acetate; (4-methoxyphenyl)methyl formate; benzaldehyde; dimethoxymethylbenzene; 4-methyl-2-phenyl-1,3-dioxolane; phenylmethyl acetate; phenylmethanol; 2-phenylethanol; phenylmethyl 3-phenylprop-2-enoate; phenylmethyl formate; phenylmethyl 2-phenylacetate; 1,7,7-trimethylbicyclo[2.2.1]heptan-6-ol; (1-methyl-2-oxo-propyl) butanoate; 2,3-dihydroxybutane; butan-1-ol; butyl 2-methylbutanoate; butyl acetate; butyl butanoate; (1-butoxy-1-oxopropan-2-yl) butanoate; butyl 3-methylbutanoate; 5-butyl-4-methyloxolan-2-one; butanoic acid; oxolan-2-one; 2-methyl-5-propan-2-ylphenol; 2-methyl-5-prop-1-en-2-ylcyclohex-2-en-1-ol; 4-methyl-1-propan-2-ylcyclohex-3-en-1-ol; 2-methyl-5-prop-1-en-2-ylcyclohex-2-en-1-one; (5 s)-2-methyl-5-prop-1-en-2-ylcyclohex-2-en-1-one; (5r)-2-methyl-5-prop-1-en-2-ylcyclohex-2-en-1-one; (1r,4e,9s)-4,11,11-trimethyl-8-methylidenebicyclo[7.2.0]undec-4-ene; 4,5-epoxy-4,11,11-trimethyl-8-methylenebicyclo(7.2.0)undecane; (e)-3-phenylprop-2-enal; 3-phenylprop-2-enoic acid; [(e)-3-phenylprop-2-enyl] acetate; [(e)-3-phenylprop-2-enyl] (e)-3-phenylprop-2-enoate; z-dec-4-enal; (z)-non-6-en-1-ol; 3,7-dimethylocta-2,6-dienal; 3,7-dimethyloct-6-enal; 3,7-dimethyloct-6-en-1-ol; 3,7-dimethyloct-6-enyl acetate; (e)-1-(2,6,6-trimethyl-1-cyclohexa-1,3-dienyl)but-2-en-1-one; (e)-1-(2,6,6-trimethyl-1-cyclohex-2-enyl)but-2-en-1-one; 1-(2,6,6-trimethyl-1-cyclohexenyl)but-2-en-1-one; 6-pentyloxan-2-one; 5-hexyloxolan-2-one; decanal; decanoic acid; diethyl propanedioate; 2,3-diethylpyrazine; diethyl decanedioate; chroman-2-one; 3-methyl-2-pentylcyclopent-2-en-1-one; 1,3-dimethoxybenzene; 1,4-dimethoxybenzene; 2,6-dimethoxyphenol; (2-methyl-1-phenylpropan-2-yl) butanoate; 2,3-dimethylpyrazine; 2,5-dimethylpyrazine; 2,6-dimethylpyrazine; methylsulfanyldisulfanylmethane; 3,4-dimethylcyclopentane-1,2-dione; 3-hydroxy-4,5-dimethyl-5h-furan-2-one; 2,6-dimethylhept-5-enal; 4-hydroxy-2,5-dimethylfuran-3-one; 2,6-dimethylpyridine; phenoxybenzene; 6-heptyloxan-2-one; 5-octyloxolan-2-one; ethyl 3-methylsulfanylpropanoate; ethyl 3-hydroxybutanoate; ethyl acetate; ethyl 3-oxobutanoate; ethyl benzoate; ethyl butanoate; ethyl 3-phenylprop-2-enoate; ethyl decanoate; ethyl formate; 4-ethyl-2-methoxyphenol; ethyl heptanoate; ethyl hexanoate; ethyl 2-methylpropanoate; ethyl 3-methylbutanoate; ethyl 2-hydroxypropanoate; ethyl dodecanoate; ethyl 4-oxopentanoate; 2-ethyl-3-hydroxypyran-4-one; ethyl 3-methyl-3-phenyloxirane-2-carboxylate; ethyl tetradecanoate; ethyl nonanoate; ethyl octanoate; ethyl (z)-octadec-9-enoate; ethyl hexadecanoate; ethyl propanoate; ethyl (e)-but-2-enoate; ethyl (e)-oct-2-enoate; ethyl pentanoate; 3-ethoxy-4-hydroxybenzaldehyde; 2-ethyl-3,5-dimethylpyrazine; 3-ethyl-2-hydroxycyclopent-2-en-1-one; ethyl 2-methylbutanoate; ethyl 2-methylpentanoate; 2-ethyl-3-methylpyrazine; 2-ethyl-3,5-dimethylpyrazine; ethyl-3-hexenoate; 5-ethyl-3-hydroxy-4-methyl-5h-furan-2-one; 5-ethyl-4-hydroxy-2-methylfuran-3-one; 4-ethylphenol; 3-ethylpyridine; 4-ethylpyridine; 4,7,7-trimethyl-8-oxabicyclo[2.2.2]octane; 2-methoxy-4-prop-2-enylphenol; (1s,4r,6s)-1,5,5-trimethylbicyclo[2.2.1]heptan-6-ol; furan-2-ylmethyl acetate; furan-2-ylmethanethiol; furan-2-ylmethyl propanoate; (2e)-3,7-dimethylocta-2,6-dien-1-ol; [(2e)-3,7-dimethylocta-2,6-dienyl] acetate; (5e)-6,10-dimethylundeca-5,9-dien-2-one; [(2e)-3,7-dimethylocta-2,6-dienyl] formate; (2r,3s,4r,5r)-2,3,4,5,6-pentahydroxyhexanoic acid; 1,3-diacetyloxypropan-2-yl acetate; 2-methoxyphenol; 5-propyloxolan-2-one; heptanoic acid; heptan-2-one; heptan-1-ol; oxacycloheptadec-7-en-2-one; 6-methyloxan-2-one; 5-ethyloxolan-2-one; hexanal; hexanoic acid; hexan-1-ol; (z)-hex-3-en-1-ol; [(z)-hex-3-enyl] acetate; hex-2-enal; hex-3-enoic acid; (e)-hex-2-enoic acid; hex-2-en-1-ol; hex-2-enyl acetate; [(z)-hex-3-enyl] butanoate; [(z)-hex-3-enyl] formate; hex-3-enyl 2-methylbutanoate; hexyl 2-methylbutanoate; hexyl acetate; hexyl butanoate; hexyl formate; hexyl hexanoate; hexyl 2-hydroxypropanoate; hexyl octanoate; (1e,4e,8e)-2,6,6,9-tetramethylcycloundeca-1,4,8-triene; 2-hydroxy-4-methylbenzaldehyde; 4-hydroxy-5-methylfuran-3-one; 7-hydroxy-3,7-dimethyloctanal; 4-(4-hydroxyphenyl)butan-2-one; 4-(2,6,6-trimethyl-1-cyclohex-2-enyl)but-3-en-2-one; 4-(2,6,6-trimethyl-1-cyclohexenyl)but-3-en-2-one; (e)-4-[(1s,5r)-2,5,6,6-tetramethyl-1-cyclohex-2-enyl]but-3-en-2-one; 3-methylbutyl acetate; 3-methylbutyl butanoate; 3-methylbutyl hexanoate; 3-methylbutyl 3-methylbutanoate; 3-methylbutyl 2-hydroxypropanoate; 3-methylbutyl octanoate; 3-methylbutyl propanoate; 3-methylbutyl pentanoate; [(1s,4r,6s)-1,7,7-trimethyl-6-bicyclo[2.2.1]heptanyl] acetate; 2-methylpropan-1-ol; 2-methylpropyl acetate; 3-oxo-butanoic acid, 2-methylpropyl ester; 2-methylpropyl butanoate; 2-methylpropyl hexanoate; 2-methylpropyl 3-methylbutanoate; 2-methylpropyl 2-methylbutanoate; 2-methylpropanal; 2-methylpropanoic acid; 1,2-dimethoxy-4-prop-1-enylbenzene; 5-methyl-2-propan-2-ylcyclohexan-1-one; propan-2-yl tetradecanoate; 5-methyl-2-prop-1-en-2-ylcyclohexan-1-ol; 3-methylbutanoic acid; 3-methyl-2-[(z)-pent-2-enyl]cyclopent-2-en-1-one; 2,6,6-trimethylcyclohex-2-ene-1,4-dione; dodecanal; (4r)-1-methyl-4-prop-1-en-2-ylcyclohexene; 3,7-dimethylocta-1,6-dien-3-ol; 2-(5-methyl-5-vinyltetrahydro-2-furanyl)-2-propanol; 3,7-dimethylocta-1,6-dien-3-yl acetate; 3,7-dimethylocta-1,6-dien-3-yl butanoate; 3-hydroxy-2-methylpyran-4-one; (4z)-4-[(e)-but-2-enylidene]-3,5,5-trimethylcyclohex-2-en-1-one; 1-methyl-4-propan-2-ylcyclohexa-1,4-diene; 5-methyl-2-(2-sulfanylpropan-2-yl)cyclohexan-1-one; (1r,2s,5r)-5-methyl-2-propan-2-ylcyclohexan-1-ol; (2s,5r)-2-isopropyl-5-methylcyclohexanone; [(6s,9r)-9-methyl-6-propan-2-yl-1,4-dioxaspiro[4.5]decan-3-yl]methanol; (5-methyl-2-propan-2-ylcyclohexyl) acetate; (5-methyl-2-propan-2-ylcyclohexyl) 2-methylbutanoate; 3-methylsulfanylpropanal; 4-methoxybenzaldehyde; 2-methoxy-3-methyl-pyrazine; 2-methoxy-4-methylphenol; 4-ethenyl-2-methoxyphenol; p-anisyl alcohol; 4-(4-methoxyphenyl)butan-2-one; 2-methyl-2-pentenoic acid; 1-(4-methylphenyl)ethanone; 1-methoxy-4-methylbenzene; methyl 2-aminobenzoate; 1-phenylethyl acetate; 2-methylbutan-1-ol; 2-methylbutyl acetate; 3-methylbutyl 2-methylpropanoate; 2-methylbutanal; 3-methylbutanal; methyl butanoate; 2-methylbutanoic acid; methyl (e)-3-phenylprop-2-enoate; 3-methylcyclopentane-1,2-dione; methyl 2-(3-oxo-2-pentylcyclopentyl)acetate; 5-methylfuran-2-carbaldehyde; 2-(methyldisulfanylmethyl)furan; methyl hexanoate; methyl 2-methylpropanoate; methyl 2-methylaminobenzoate; methyl 4-methoxybenzoate; 3-methylpentanoic acid; methyl 2-phenylacetate; 2-methylpyrazine; 5-methylquinoxaline; methylsulfanylmethane; 2-methyloxolan-3-one; s-methyl butanethioate; methyl (e)-non-2-enoate; 2-methylpentanoic acid; 3-methylcyclohexane-1,2-dione; methyl furan-2-carboxylate; methyl 2-methylbutanoate; 1-(1h-pyrrol-2-yl)ethanone; methyl 3-methylsulfanylpropanoate; 6-methylhepta-3,5-dien-2-one; 6-methylhept-5-en-2-one; 2-(4-methyl-1,3-thiazol-5-yl)ethanol; 5-methyl-6,7-dihydro-5h-cyclopenta[b]pyrazine; (e)-1-(2,6,6-trimethyl-1-cyclohex-2-enyl)pent-1-en-3-one; 3-methylbutan-1-ol; 3-methylpyridine; 4-methylpyridine; 6-methylquinoline; 5-methylthiophene-2-carbaldehyde; (e)-2-methylbut-2-enoic acid; (1s,2s,5r)-5-methyl-2-propan-2-ylcyclohexan-1-ol; (2z)-3,7-dimethylocta-2,6-dien-1-ol; 3,7,11-trimethyldodeca-1,6,10-trien-3-ol; [(2z)-3,7-dimethylocta-2,6-dienyl] acetate; (2e,6z)-nona-2,6-dienal; 2,6-nonadien-1-ol; 6-butyloxan-2-one; 5-pentyltetrahydrofuran-2-one; nonanal; nonanoic acid; nonan-2-one; (z)-non-6-enal; (3e)-3,7-dimethylocta-1,3,6-triene; 6-propyloxan-2-one; 5-butyloxolan-2-one; octanal; octanoic acid; oct-1-en-3-ol; octyl acetate; (e)-octadec-9-enoic acid; 5-methyl-3h-furan-2-one; 1-oxacyclohexadecan-2-one; pentan-1-ol; pentan-2-one; 2-phenylethyl 3-methylbutanoate; 2-phenylethyl 2-phenylacetate; 2-phenylacetaldehyde; 2-phenylacetic acid; 3-phenylpropanoic acid; 3-phenylpropan-1-ol; 2-phenyl-2-butenal; 3-phenyl-2-propen-1-ol; 4,7,7-trimethylbicyclo[3.1.1]hept-3-ene; 7,7-dimethyl-4-methylidenebicyclo[3.1.1]heptane; (6s)-3-methyl-6-propan-2-ylcyclohex-2-en-1-one; 1,3-benzodioxole-5-carbaldehyde; 2-ethoxy-5-[(e)-prop-1-enyl]phenol; propanoic acid; propyl acetate; propyl butanoate; propyl formate; 3-propylidene-2-benzofuran-1-one; 2-oxopropanoic acid; 3,7-dimethyloct-6-en-1-ol; (2r,3r)-2,3-dihydroxysuccinic acid; 2-[(1s)-4-methyl-1-cyclohex-3-enyl]propan-2-ol; 1-methyl-4-propan-2-ylidenecyclohexene; 2-(4-methyl-1-cyclohex-3-enyl)propan-2-yl acetate; 5,6,7,8-tetrahydroquinoxaline; 2,3,5,6-tetramethylpyrazine; 2,6,6,10-tetramethyl-1-oxaspiro[4.5] dec-9-ene; 5-methyl-2-propan-2-ylphenol; (4-methylphenyl) acetate; 4-methylbenzaldehyde; (4-methylphenyl) 3-methylbutanoate; e-2-methoxy-4-prop-1-enylphenol; 2,3,5-trimethylphenol; 2,3,5-trimethylpyrazine; 6-hexyloxan-2-one; 5-heptyloxolan-2-one; undecan-2-one; pentanal; pentanoic acid; 5-methyloxolan-2-one; 4-hydroxy-3-methoxybenzaldehyde; 3,4-dimethoxybenzaldehyde; 2-phenylethyl acetate; (e)-hex-2-enoic acid; (3ar,5as,9as,9br)-3a,6,6,9a-tetramethyldodecahydronaphtho[2,1-b]furan; 6-pentyl-5,6-dihydropyran-2-one; 2-phenylethyl 2-methylpropanoate; 2-methyl-1[1-(2-methylbutoxy)ethoxy]butane; 4,4a-dimethyl-6-prop-1-en-2-yl-3,4,5,6,7,8-hexahydronaphthalen-2-one; 2-(1-Mercapto-1-methylethyl)-5-methylcyclohexanone; and mixtures thereof

The one or more flavors may be selected from 4-(4-Methoxyphenyl)-2-butanone [also known as (4-(para-)methoxyphenyl)-2-butanone], 4-Hydroxy-3-methoxybenzaldehyde [also known as vanillin], 5-heptyloxolan-2-one [also known as γ-undecalactone], (2S,5R)-2-Isopropyl-5-methylcyclohexanone [also known as menthone], 2-ethoxy-5-[(E)-prop-1-enyl]phenol [also known as 5-propenyl guaethol], (1R,2S,5R)-5-methyl-2-propan-2-ylcyclohexan-1-ol [also known as menthol], 2-(1-Mercapto-1-methylethyl)-5-methylcyclohexanone [also known as para-mentha-8-thiol-3-one] and mixtures thereof. In one aspect the flavorflavour is at least menthol.

If present, the one or more flavors may be present in any suitable amount. In one aspect the one or more flavors are present in a total amount of no greater than 10 wt. % based on the aerosolizable formulation. In one aspect the one or more flavors are present in a total amount of no greater than 7 wt. % based on the aerosolizable formulation. In one aspect the one or more flavors are present in a total amount of no greater than 5 wt. % based on the aerosolizable formulation. In one aspect the one or more flavors are present in a total amount of no greater than 4 wt. % based on the aerosolizable formulation. In one aspect the one or more flavors are present in a total amount of no greater than 3 wt. % based on the aerosolizable formulation. In one aspect the one or more flavors are present in a total amount of no greater than 2 wt. % based on the aerosolizable formulation. In one aspect the one or more flavors are present in a total amount of no greater than 1 wt. % based on the aerosolizable formulation.

In one aspect the one or more flavors are present in a total amount of from 0.01 to 5 wt. % based on the aerosolizable formulation. In one aspect the one or more flavors are present in a total amount of from 0.01 to 4 wt. % based on the aerosolizable formulation. In one aspect the one or more flavors are present in a total amount of from 0.01 to 3 wt. % based on the aerosolizable formulation. In one aspect the one or more flavors are present in a total amount of from 0.01 to 2 wt. % based on the aerosolizable formulation. In one aspect the one or more flavors are present in a total amount of from 0.01 to 1 wt. % based on the aerosolizable formulation. In one aspect the one or more flavors are present in a total amount of from 0.01 to 0.5 wt. % based on the aerosolizable formulation.

Encapsulating Material

The aerosolizable formulation comprises one or more encapsulating materials. The one or more encapsulating materials may be present in any suitable amount in the aerosolizable formulation. In one aspect the one or more encapsulating materials are present in a total amount of no greater than 12 wt. % based on the aerosolizable formulation. In one aspect the one or more encapsulating materials are present in a total amount of no greater than 10 wt. % based on the aerosolizable formulation. In one aspect the one or more encapsulating materials are present in a total amount of no greater than 9 wt. % based on the aerosolizable formulation. In one aspect the one or more encapsulating materials are present in a total amount of no greater than 8 wt. % based on the aerosolizable formulation. In one aspect the one or more encapsulating materials are present in a total amount of no greater than 7 wt. % based on the aerosolizable formulation. In one aspect the one or more encapsulating materials are present in a total amount of no greater than 6 wt. % based on the aerosolizable formulation. In one aspect the one or more encapsulating materials are present in a total amount of no greater than 5 wt. % based on the aerosolizable formulation. In one aspect the one or more encapsulating materials are present in a total amount of no greater than 4 wt. % based on the aerosolizable formulation. In one aspect the one or more encapsulating materials are present in a total amount of no greater than 3 wt. % based on the aerosolizable formulation. In one aspect the one or more encapsulating materials are present in a total amount of no greater than 2 wt. % based on the aerosolizable formulation. In one aspect the one or more encapsulating materials are present in a total amount of no greater than 1 wt. % based on the aerosolizable formulation. In one aspect the one or more encapsulating materials are present in a total amount of no greater than 0.1 wt. % based on the aerosolizable formulation. In one aspect the one or more encapsulating materials are present in a total amount of no greater than 0.01 wt. % based on the aerosolizable formulation. In one aspect the one or more encapsulating materials are present in a total amount of no greater than 0.001 wt. % based on the aerosolizable formulation.

In one aspect the one or more encapsulating materials is selected from the group consisting of micelles, cyclodextrins, calixarenes, metal organic frameworks, dendrimers, polymers, hydrocolloids, pollen spores, yeast particles, porous silica, and mixtures thereof. In one aspect the one or more encapsulating materials are selected from cyclodextrins and mixtures thereof.

The one or more cyclodextrins may be selected from the group consisting of unsubstituted cyclodextrins, substituted cyclodextrins and mixtures thereof. In one aspect at least one cyclodextrin is an unsubstituted cyclodextrin. In one aspect the one or more cyclodextrins are selected from the group consisting of unsubstituted cyclodextrins. In one aspect at least one cyclodextrin is a substituted cyclodextrin. In one aspect the one or more cyclodextrins are selected from the group consisting of substituted cyclodextrins.

In one aspect the one or more cyclodextrins are selected from the group consisting of unsubstituted (α)-cyclodextrin, substituted (α)-cyclodextrin, unsubstituted (β)-cyclodextrin, substituted (β)-cyclodextrin, unsubstituted (γ)-cyclodextrin, substituted (γ)-cyclodextrin, and mixtures thereof. In one aspect the one or more cyclodextrins are selected from the group consisting of unsubstituted (β)-cyclodextrin, substituted (β)-cyclodextrin, and mixtures thereof.

In one aspect the one or more cyclodextrins are selected from the group consisting of unsubstituted (α)-cyclodextrin, unsubstituted (β)-cyclodextrin, unsubstituted (γ)-cyclodextrin, and mixtures thereof. In one aspect the one or more cyclodextrins is selected from unsubstituted (β)-cyclodextrin.

In one aspect the one or more cyclodextrins are selected from the group consisting of substituted (α)-cyclodextrin, substituted (β)-cyclodextrin, substituted (γ)-cyclodextrin, and mixtures thereof. In one aspect the one or more cyclodextrins is selected from substituted (β)-cyclodextrins. Chemical substitutions at the 2-, 3-, and 6-hydroxyl sites are envisaged, and in particular substitution at the 2-position.

In one aspect the one or more cyclodextrins are selected from the group consisting of 2-hydroxy-propyl-α-cyclodextrin, 2-hydroxy-propyl-β-cyclodextrin, 2-hydroxy-propyl-γ-cyclodextrin and mixtures thereof. In one aspect the one or more cyclodextrins is at least 2-hydroxy-propyl-α-cyclodextrin. In one aspect the one or more cyclodextrins is at least 2-hydroxy-propyl-O-cyclodextrin. In one aspect the one or more cyclodextrins is at least 2-hydroxy-propyl-γ-cyclodextrin.

2-hydroxy-propyl derivatives of cyclodextrins, such as 2-hydroxy-propyl-β-cyclodextrin have increased solubility in water when compared to base cyclodextrins such as β-cyclodextrin.

One or more cyclodextrins may or may not be present in any suitable amount in the aerosolizable formulation. In one aspect the one or more cyclodextrins are present in a total amount of no greater than 12 wt. % based on the aerosolizable formulation. In one aspect the one or more cyclodextrins are present in a total amount of no greater than 10 wt. % based on the aerosolizable formulation. In one aspect the one or more cyclodextrins are present in a total amount of no greater than 9 wt. % based on the aerosolizable formulation. In one aspect the one or more cyclodextrins are present in a total amount of no greater than 8 wt. % based on the aerosolizable formulation. In one aspect the one or more cyclodextrins are present in a total amount of no greater than 7 wt. % based on the aerosolizable formulation. In one aspect the one or more cyclodextrins are present in a total amount of no greater than 6 wt. % based on the aerosolizable formulation. In one aspect the one or more cyclodextrins are present in a total amount of no greater than 5 wt. % based on the aerosolizable formulation. In one aspect the one or more cyclodextrins are present in a total amount of no greater than 4 wt. % based on the aerosolizable formulation. In one aspect the one or more cyclodextrins are present in a total amount of no greater than 3 wt. % based on the aerosolizable formulation. In one aspect the one or more cyclodextrins are present in a total amount of no greater than 2 wt. % based on the aerosolizable formulation. In one aspect the one or more cyclodextrins are present in a total amount of no greater than 1 wt. % based on the aerosolizable formulation. In one aspect the one or more cyclodextrins are present in a total amount of no greater than 0.1 wt. % based on the aerosolizable formulation. In one aspect the one or more cyclodextrins are present in a total amount of no greater than 0.01 wt. % based on the aerosolizable formulation. In one aspect the one or more cyclodextrins are present in a total amount of no greater than 0.001 wt. % based on the aerosolizable formulation.

In one aspect if the aerosolizable formulation contains one or more cyclodextrins, then the aerosolizable formulation contains no flavors that can be encapsulated by the one or more cyclodextrins. In one aspect if the aerosolizable formulation contains one or more cyclodextrins, then the aerosolizable formulation contains no flavors.

As discussed herein, in one aspect the present disclosure provides an aerosolizable formulation comprising (i) water (ii) one or more flavors to be encapsulated; and (iii) one or more encapsulating materials having a solubility in water of at least 50% of the solubility in water of the one or more flavors to be encapsulated; and (iv) nicotine.

In one aspect, the one or more encapsulating materials have a solubility in water of at least 55% of the solubility in water of the one or more flavors to be encapsulated. In one aspect, the one or more encapsulating materials have a solubility in water of at least 60% of the solubility in water of the one or more flavors to be encapsulated. In one aspect, the one or more encapsulating materials have a solubility in water of at least 65% of the solubility in water of the one or more flavors to be encapsulated. In one aspect, the one or more encapsulating materials have a solubility in water of at least 70% of the solubility in water of the one or more flavors to be encapsulated. In one aspect, the one or more encapsulating materials have a solubility in water of at least 75% of the solubility in water of the one or more flavors to be encapsulated. In one aspect, the one or more encapsulating materials have a solubility in water of at least 80% of the solubility in water of the one or more flavors to be encapsulated. In one aspect, the one or more encapsulating materials have a solubility in water of at least 85% of the solubility in water of the one or more flavors to be encapsulated. In one aspect, the one or more encapsulating materials have a solubility in water of at least 90% of the solubility in water of the one or more flavors to be encapsulated. In one aspect, the one or more encapsulating materials have a solubility in water of at least 95% of the solubility in water of the one or more flavors to be encapsulated.

Encapsulating Material and Flavorflavour

The one or more encapsulating materials and the flavor may be present in any suitable amount relative to each other. The molar ratio of encapsulating material to flavor may be selected as desired. In one aspect the molar ratio of encapsulating material to flavor is from 5:1 to 1:5. In one aspect the molar ratio of encapsulating material to flavor is from 4:1 to 1:4. In one aspect the molar ratio of encapsulating material to flavor is from 3:1 to 1:3. In one aspect the molar ratio of encapsulating material to flavor is from 2:1 to 1:2. In one aspect the molar ratio of encapsulating material to flavor is from 1.5:1 to 1:1.5. In one aspect the molar ratio of encapsulating material to flavor is from 1.2:1 to 1:1.2. In one aspect the molar ratio of encapsulating material to flavor is from 5:1 to 1:1. In one aspect the molar ratio of encapsulating material to flavor is from 4:1 to 1:1. In one aspect the molar ratio of encapsulating material to flavor is from 3:1 to 1:1. In one aspect the molar ratio of encapsulating material to flavor is from 2:1 to 1:1. In one aspect the molar ratio of encapsulating material to flavor is from 1.5:1 to 1:1. In one aspect the molar ratio of encapsulating material to flavor is from 1.4:1 to 1:1. In one aspect the molar ratio of encapsulating material to flavor is from 1.3:1 to 1:1. In one aspect the molar ratio of encapsulating material to flavor is from 1.2:1 to 1:1. In one aspect the molar ratio of encapsulating material to flavor is from 1.1:1 to 1:1. In one aspect the molar ratio of encapsulating material to flavor is approximately 1:1.

Process

As discussed herein, in one aspect there is provided a process for improving the sensory properties of an aerosolized nicotine formulation, the process comprising the steps of aerosolizing an aerosolizable formulation comprising
(i) water;
(ii) one or more flavors to be encapsulated; and
(iii) one or more encapsulating materials;

wherein in the presence of water the energy of binding of the one or more encapsulating materials with the one or more flavors to be encapsulated is from −0.5 to −8 kcal/mol.

As discussed herein, in one aspect there is provided a process for forming an aerosol, the process comprising aerosolizing an aerosolizable formulation comprising (i) water (ii) one or more flavors to be encapsulated; and (iii) one or more encapsulating materials; wherein in the presence of water the energy of binding of the one or more encapsulating materials with the one or more flavors to be encapsulated is from −0.5 to −8 kcal/mol.

In the process the aerosol may be formed by a process performed at a temperature below 60° C. In the process the aerosol may be formed by a process performed at a temperature below 50° C. In the process the aerosol may be formed by a process performed at a temperature below 40° C. In the process the aerosol may be formed by a process performed at a temperature below 30° C. In the process the aerosol may be formed by a process performed at a temperature below 25° C. In the process the aerosol may be formed by a process which does not involve heating.

In the process the aerosol may be formed by applying ultrasonic energy to the aerosolizable formulation.

In one aspect the aerosol the aerosol of the aerosolized formulation has a D50 of from 2 to 6 μm. References in the present specification to particle size distribution, D50, D10 or D90 refer to values measured in accordance with British and European Pharmacopoeia, 2.9.31 Particle Size Analysis By Laser Light Diffraction (see BRITISH PHARMACOPOEIA COMMISSION. (2014), British Pharmacopoeia. London, England: Stationery Office and COUNCIL OF EUROPE. (2013). European Pharmacopoeia. Strasbourg, France: Council of Europe). The terms D50, Dv50 and Dx50 are interchangeable. The terms D10, Dv10 and Dx10 are interchangeable. The terms D90, Dv90 and Dx90 are interchangeable.

In one aspect the aerosol has a D50 of from 2.5 to 6 μm. In one aspect the aerosol has a D50 of from 3 to 6 μm. In one aspect the aerosol has a D50 of from 3.5 to 6 μm. In one aspect the aerosol has a D50 of from 4 to 6 μm. In one aspect the aerosol has a D50 of from 4.5 to 6 μm.

In one aspect the aerosol has a D50 of from 5 to 6 μm. In one aspect the aerosol has a D50 of from 2.5 to 5.5 μm. In one aspect the aerosol has a D50 of from 3 to 5.5 μm. In one aspect the aerosol has a D50 of from 3.5 to 5.5 μm. In one aspect the aerosol has a D50 of from 4 to 5.5 μm. In one aspect the aerosol has a D50 of from 4.5 to 5.5 μm. In one aspect the aerosol has a D50 of from 5 to 5.5 μm.

In one aspect the aerosol has a D10 of at least 0.5 μm. In one aspect the aerosol has a D10 of at least 1 μm. In one aspect the aerosol has a D10 of at least 2 μm.

In one aspect the aerosol has a D90 of no greater than 15 μm. In one aspect the aerosol has a D90 of no greater than 12 μm. In one aspect the aerosol has a D90 of no greater than 10 μm.

In one aspect D50 is measured after exclusion of particles having a particle size of less than 1 μm. In one aspect D10 is measured after exclusion of particles having a particle size of less than 1 μm. In one aspect D90 is measured after exclusion of particles having a particle size of less than 1 μm.

The formulation may be contained or delivered by any means. In one aspect the present disclosure provides a contained aerosolizable formulation comprising (a) one or more containers; and (b) an aerosolizable formulation as defined herein. The container may be any suitable container, for example to allow for the storage or delivery of the formulation. In one aspect the container is configured for engagement with an electronic aerosol provision system. The container may be configured to become fluidly in communication with an electronic aerosol provision system so that formulation may be delivered to the electronic aerosol provision system. As described above, the present disclosure relates to container which may be used in an electronic aerosol provision system, such as an e-cigarette. Throughout the following description the term “e-cigarette” is used; however, this term may be used interchangeably with electronic aerosol provision system.

As discussed herein, the container of the present disclosure is typically provided for the delivery of aerosolizable formulation to or within an e-cigarette. The aerosolizable formulation may be held within an e-cigarette or may be sold as a separate container for subsequent use with or in an e-cigarette. As understood by one skilled in the art, e-cigarettes may contain a unit known as a detachable cartomizer which typically comprises a reservoir of aerosolizable formulation, an aerosolizer such as a wick material and a heating element for vaporizing the aerosolizable formulation. In some e-cigarettes, the cartomizer is part of a single-piece device and is not detachable. In one aspect the container is a cartomizer or is part of a cartomizer. In one aspect the container is not a cartomizer or part of a cartomizer and is a container, such as a tank, which may be used to deliver nicotine formulation to or within an e-cigarette.

In one aspect the container is part of an e-cigarette. Therefore in a further aspect the present disclosure provides an electronic aerosol provision system comprising: an aerosolizable formulation as defined herein; an aerosolizer for aerosolizing formulation for inhalation by a user of the electronic aerosol provision system; and a power supply comprising a cell or battery for supplying power to the aerosolizer.

In addition to the aerosolizable formulation of the present disclosure and to systems such as containers and electronic aerosol provision systems containing the same, the present disclosure provides a process for improving the sensory properties of an aerosolized nicotine.

Reference to an improvement in the sensory properties of a vaporized nicotine solution refer may include an improvement in the smoothness of the vaporized nicotine solution as perceived by a user.

The process of the present disclosure may comprises additional steps either before the steps listed, after the steps listed or between one or more of the steps listed.

The disclosure will now be described with reference to the following non-limiting example.

### Example

The binding and energy of binding of hydroxypropyl beta cyclodextrin and menthol was studied with automated docking simulations. Automated docking simulations use a three-dimensional representation to assess the fit for a guest substrate in a molecular cavity. It is generally accepted that the predicted binding energies may be accurately determined with automated docking simulations.

Docking was performed in Autodock 4.2 [The Scripps Research Institute, La Jolla, Calif., USA] (Morris, et al., 2009) using settings as detailed below. In brief, hydrogens were merged and Kolman/Gasteiger charges were added according to standard methodology. Grid size was adjusted where necessary to accommodate the receptor. In general, settings were left at default values. The receptors were treated as rigid entities. Output format was Lamarckian genetic algorithm.

Host—Hydroxypropyl Beta Cyclodextrin

Hydroxypropyl-beta-CD (HP-β-CD) contains numerous isomers, due to random substitution during synthesis. The primary hydroxyl groups at the C-6 of the sugars are the most likely to be substituted owing to their nucleophilicity and lack of steric crowding, but substitution can also occur at the C-2 and C-3 positions, which are at the opposite face of the cavity to C-6. The crystal structures of beta (β) cyclodextrin were taken from The Cambridge Crystallographic Data Centre (CCDC) (item designation “ARUXIU”) and modified with hydroxypropyl groups according to several substitution patterns to see how important this is to binding.

The following variants were trialed:

Version 1: No substitution of the beta cyclodextrin

Version 2: Five (out of seven) of the C-6 hydroxyls were functionalized, along with one C-2 and one C-3, all selected at random

Version 3: Five of the C-6 hydroxyls were functionalized, selected at random

These modifications were made using Discovery Studio Visualizer [v16.1.0.15350, (2015), Dassault Systémes Biovar Corp]. The structures were then optimized with the fast, Dreiding-like force field tool within Discovery Studio Visualizer and converted to .pdb format for onward processing using Autodock 4.2. The settings used in the modelling each of Versions 1, 2 and 3 are provided in the table below.

| | Further | Simulation version | | |
|---|---|---|---|---|
| Setting | information | 1 | 2 | 3 |
| Grid Point Spacing | Angstroms | 0.375 | 0.375 | 0.375 |
| Number of grid points in each | x | 40 | 40 | 50 |
| Cartesian direction | y | 40 | 40 | 40 |
| | z | 40 | 40 | 50 |
| Coordinates of Central Grid | | (4.422, 6.023, | (4.793, 6.323, | (4.445, 6.360, |
| Point of Map | | 14.126) | 14.339) | 14.413) |
| Minimum coordinates in grid | | (−3.078, −1.477, | (−2.707, −1.177, | (−4.930, −1.140, |
| | | 6.626) | 6.839) | 5.038) |
| Maximum coordinates in grid | | (11.922, 13.523, | (12.293, 13.823, | (13.820, 13.860, |
| | | 21.626) | 21.839) | 23.788) |

| | | |
|---|---|---|
| User-specified initial position | | random |
| for ligand | | |
| Initial relative dihedral offset | | random |
| User-specified initial relative | | random |
| dihedrals | | |
| Docking search parameter | | Genetic Algorithm |
| Number of requested GA | | 10 runs |
| dockings | | |
| Population size | | 150 |
| Maximum number of | | 2500000 |
| evaluations | | |
| Maximum number of top | | 1 |
| individuals that | | |
| automatically survive | | |
| Rate of gene mutation | | 0.02 |
| Rate of crossover | | 0.08 |
| GA crossover mode | | “twopt” |
| Mean of Cauchy distribution | alpha | 0 |
| for gene mutation | parameter | |
| Variance of Cauchy | beta | 1 |
| distribution for gene mutation | parameter | |
| Number of generations fro | | 10 |
| picking worst individuals | | |
| Docking output | | Lamarckian GA |

Guest—Menthol

The 3-dimensional structure for menthol was obtained from Pubchem and converted to .pdb format for use in docking simulation.

Docking Results

The docking simulation defaults to 10 repetitions to check for viable conformations between host and guest. Therefore, each simulation gives 10 results. The result is expressed in terms of Gibbs free energy of binding. A negative value denotes an energetically favored process. The absolute size of this binding energy is a useful comparator of binding affinity. As a general rule, negative values with an absolute size above around 5 Kcal/mole indicate moderately strong binding affinity. The full data for each version are given in docking log files herein but are summarized below, along with an image showing a representative binding complex for each version.

Version 1: Unsubstituted Beta Cyclodextrin

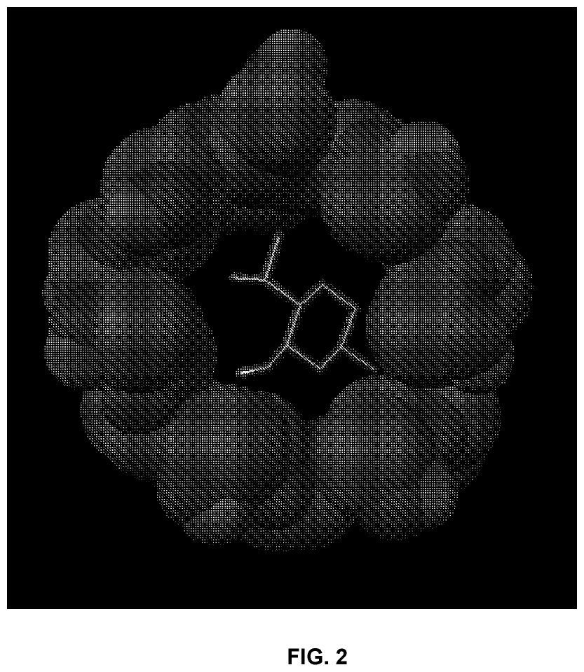

The docking simulation gave several conformations, in all cases binding the guest within the cavity of the host—as typified by FIG. 2, which shows atomic spheres for the host and a line representation for the guest to improve clarity. The corresponding docking log file is named menBCD.dlg.

The runs provided a free energy of binding of −5.1 Kcal/mole.

Version 2: Hydroxypropyl Beta Cyclodextrin Substituted at 7 Sites

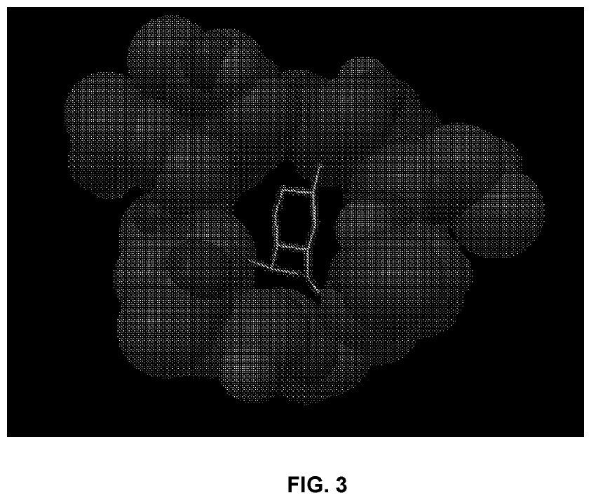

The docking simulation gave several conformations, in all cases binding the guest within the cavity of the host—as typified by FIG. 3, which shows atomic spheres for the host and a line representation for the guest to improve clarity.

The runs provided a free energy of binding of −6.0 Kcal/mole.

Version 3: Hydroxypropyl Beta Cyclodextrin Substituted at 5 Points

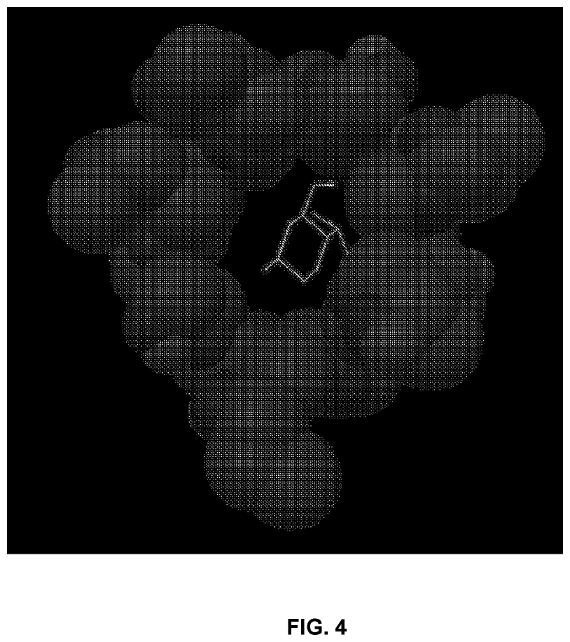

The docking simulation gave several conformations, in all cases binding the guest within the cavity of the host—as typified by FIG. 4, which shows atomic spheres for the host and a line representation for the guest to improve clarity.

The runs provided a free energy of binding of −5.6 Kcal/mole.

Summary and Conclusions

The binding of menthol in hydroxypropyl beta cyclodextrin can be modelled using molecular docking, which establishes a binding free energy of between −5 and −6 Kcal/mole.

The extent and location of the hydroxypropyl groups in HP-β-CD can vary from molecule to molecule. We therefore checked the docking process using a range of possible structures to ensure sensitivity of the measurement towards fluctuations in structure. Two versions of the hydroxypropyl derivative of cyclodextrin were assessed, and gave broadly similar results. An unfunctionalized variant was also assessed, and gave reasonably similar results.

Various modifications and variations of the present invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in chemistry or related fields are intended to be within the scope of the following claims.

Aspects of the disclosure are also provided in which Hansen sphere values for one or more encapsulating materials are selected to achieve desirable compatibility with flavors included in the aerosolizable formulation, and with nicotine, if present. As will be understood by one skilled in the art Hansen sphere values describe the interaction between flavors and the encapsulating material. The relevant measurements are:

δD—Measure of dispersion forces

δP—Measure of polar (dipolar) interactions

δH—Measure of hydrogen bonding

The closer these values are for flavor and solvent (water) the more soluble they will be. Through selection of the Hansen sphere values the encapsulating material encapsulates at least one of the one or more flavors in preference to other components such as nicotine.

As will be understood by one skilled in the art compatibility between encapsulating material (host) and the encapsulated species (guest) can be defined as how “alike” they are. This can also be measured using the Hansen Solubility Parameters (HSP) Distance (also termed Ra in the equation below) between the host and guest molecules.

Ra²=4(δD₁−δD₂)²+(δP₁−δP₂)²+(δH₁−δH₂)²

where,

Ra=HSP Distance

δD=The energy from dispersion forces between molecules

δP=The energy from dipolar intermolecular force between molecules

δH=The energy from hydrogen bonds between molecules

Likeness can then be determined using the Ra of the system and an interaction radius of the guest molecule (termed Ri) as shown below:

$$R E D = \frac{R a}{R i}$$

where,

RED=Relative Energy Difference of the system

Ra=HSP Distance

Ri=Ra=HSP Distance

The RED between the encapsulating material (host) and water must be less than 1 for the encapsulating material (host) to dissolve in water. The RED between the flavor and water must be less than 1 for the flavor to dissolve in water; or the RED between the flavor and the encapsulating material (host) must be less than 1 for the flavor to be taken up by the encapsulating material (host).

Further aspects of the disclosure are described in the following numbered paragraphs:

1. An aerosolizable formulation comprising

(i) water

(ii) one or more flavors to be encapsulated;

(iii) one or more encapsulating materials; and

(iv) nicotine.

wherein (a) the water and the one or more encapsulating materials have a relative energy difference (RED) of less than 1; and (b) the one or more flavors to be encapsulated and either the water or the one or more encapsulating materials have a relative energy difference (RED) of less than 1.

2. An aerosolizable formulation comprising

(i) water

(ii) one or more flavors to be encapsulated; and

(iii) one or more encapsulating materials having a solubility in water of at least 50% of the solubility in water of the one or more flavors to be encapsulated; and

(iv) nicotine.

3. An aerosolizable formulation according to paragraph 2 wherein (a) the water and the one or more encapsulating materials have a relative energy difference (RED) of less than 1; and (b) the one or more flavors to be encapsulated and either the water or the one or more encapsulating materials have a relative energy difference (RED) of less than 1.

4. An aerosolizable formulation according to any one of paragraphs 1 to 3 wherein the one or more flavors to be encapsulated and the water have a relative energy difference (RED) of less than 1.

5. An aerosolizable formulation according to any one of paragraphs 1 to 4 wherein the one or more flavors to be encapsulated and the one or more encapsulating materials have a relative energy difference (RED) of less than 1.

6. An aerosolizable formulation according to any one of paragraphs 1 to 5 wherein the one or more flavors to be encapsulated and the water have a relative energy difference (RED) of less than 1; and wherein the one or more flavors to be encapsulated and the one or more encapsulating materials have a relative energy difference (RED) of less than 1.

7. An aerosolizable formulation according to any one of paragraphs 1 to 6 wherein water is present in an amount of at least 75 wt. % based on the aerosolizable formulation.

8. An aerosolizable formulation according to any one of paragraphs 1 to 7 wherein water is present in an amount of at least 90 wt. % based on the aerosolizable formulation.

9. An aerosolizable formulation according to any one of paragraphs 1 to 8 wherein the nicotine is present in an amount of no greater than 1 wt. % based on the aerosolizable formulation.

10. An aerosolizable formulation according to paragraph 9 wherein nicotine is present in an amount of from 0.01 to 0.6 wt. % based on the aerosolizable formulation.

11. An aerosolizable formulation according to any one of paragraphs 1 to 10 further comprising at least one acid.

12. An aerosolizable formulation according to paragraph 11 wherein the acid is selected from the group consisting of acetic acid, lactic acid, formic acid, citric acid, benzoic acid, pyruvic acid, levulinic acid, succinic acid, tartaric acid, sorbic acid, propionic acid, phenylacetic acid, and mixtures thereof.

13. An aerosolizable formulation according to paragraph 11 or 12 wherein the acid is selected from the group consisting of citric acid, benzoic acid, levulinic acid, sorbic acid, lactic acid, and mixtures thereof.

14. An aerosolizable formulation according to any one of paragraphs 11 to 13 wherein the acid is at least citric acid.

15. An aerosolizable formulation according to any one of paragraphs 11 to 14 wherein the total content of acid present in the formulation is no greater than 1.0 mole equivalents based on the nicotine.

16. An aerosolizable formulation according to any one of paragraphs 11 to 15 wherein the total content of acid present in the solution is no less than 0.1 mole equivalents based on the nicotine.

17. An aerosolizable formulation according to any one of paragraphs 1 to 16 wherein the one or more flavors are selected from the group consisting of (4-(para-)methoxyphenyl)-2-butanone, vanillin, γ-undecalactone, menthone, 5-propenyl guaethol, menthol, para-mentha-8-thiol-3-one and mixtures thereof.

18. An aerosolizable formulation according to paragraph 17 wherein the flavorflavour is at least menthol.

19. An aerosolizable formulation according to any one of paragraphs 1 to 18 wherein the one or more flavors are present in a total amount of no greater than 2 wt. % based on the aerosolizable formulation.

20. An aerosolizable formulation according to any one of paragraphs 1 to 19 wherein the one or more flavors are present in a total amount of from 0.01 to 1 wt. % based on the aerosolizable formulation.

21. An aerosolizable formulation according to any one of paragraphs 1 to 20 wherein the one or more encapsulating materials are selected from cyclodextrins and mixtures thereof.

22. An aerosolizable formulation according to paragraph 21 wherein the one more cyclodextrins are selected from the group consisting of substituted or unsubstituted (α)-cyclodextrin, substituted or unsubstituted (β)-cyclodextrin, substituted or unsubstituted (γ)-cyclodextrin, and mixtures thereof.

23. An aerosolizable formulation according to 21 or 22 wherein the one more cyclodextrins is at least a substituted (β)-cyclodextrin.

24. An aerosolizable formulation according to any one of paragraphs 1 to 23 wherein the one or more encapsulating materials are present in a total amount of no greater than 12 wt. % based on the aerosolizable formulation.

25. An aerosolizable formulation according to any one of paragraphs 1 to 24 wherein the one or more encapsulating materials have a solubility in water of at least 70% of the solubility in water of the one or more flavors to be encapsulated.

26. An aerosolizable formulation according to any one of paragraphs 1 to 25 wherein the one or more encapsulating materials have a solubility in water of at least 90% of the solubility in water of the one or more flavors to be encapsulated.

27. An aerosolizable formulation according to any one of paragraphs 1 to 25 wherein if the aerosolizable formulation contains one or more cyclodextrins, then the aerosolizable formulation contains no flavors that can be encapsulated by the one or more cyclodextrins.

28. A process for forming an aerosol, the process comprising aerosolizing an aerosolizable formulation comprising
(i) water
(ii) one or more flavors to be encapsulated; and
(iii) one or more encapsulating materials;

wherein (a) the water and the one or more encapsulating materials have a relative energy difference (RED) of less than 1; and (b) the one or more flavors to be encapsulated and either the water or the one or more encapsulating materials have a relative energy difference (RED) of less than 1.

29. A process according to paragraph 28 wherein the aerosolizable formulation is a formulation as defined in any one of paragraphs 2 to 26.

30. A process according to paragraph 28 or 29 wherein the aerosol is formed by a process performed at a temperature below 50° C.

31. A process according to paragraph 28, 29 or 30 wherein the aerosol is formed by applying ultrasonic energy to the aerosolized formulation.

32. A contained aerosolizable formulation comprising

(a) a container; and

(b) an aerosolizable formulation comprising
(i) water
(ii) one or more flavors to be encapsulated;
(iii) one or more encapsulating materials; and
(iv) nicotine;

wherein (a) the water and the one or more encapsulating materials have a relative energy difference (RED) of less than 1; and (b) the one or more flavors to be encapsulated and either the water or the one or more encapsulating materials have a relative energy difference (RED) of less than 1.

33. A contained aerosolizable formulation according to paragraph 32 wherein the container is configured for engagement with an electronic aerosol provision system.

34. An electronic aerosol provision system comprising:

(a) an aerosolizer for aerosolizing formulation for inhalation by a user of the electronic aerosol provision system;

(b) a power supply comprising a cell or battery for supplying power to the aerosolizer

(c) an aerosolizable formulation comprising
(i) water
(ii) one or more flavors to be encapsulated; and
(iii) one or more encapsulating materials;

wherein (a) the water and the one or more encapsulating materials have a relative energy difference (RED) of less than 1; and (b) the one or more flavors to be encapsulated and either the water or the one or more encapsulating materials have a relative energy difference (RED) of less than 1.

35. A process for improving the sensory properties of an aerosolized formulation, the process comprising the steps of aerosolizing an aerosolizable formulation comprising
(i) water;
(ii) one or more flavors to be encapsulated; and
(iii) one or more encapsulating materials;

wherein (a) the water and the one or more encapsulating materials have a relative energy difference (RED) of less than 1; and (b) the one or more flavors to be encapsulated and either the water or the one or more encapsulating materials have a relative energy difference (RED) of less than 1.

1. An aerosolizable formulation comprising:
(i) water present in an amount of at least 70 wt. % based on the aerosolizable formulation;
(ii) one or more flavors to be encapsulated;
(iii) one or more encapsulating materials; and
(iv) nicotine;
wherein in the presence of water an energy of binding of the one or more encapsulating materials with the one or more flavors to be encapsulated is from −0.5 to −8 kcal/mol.

2. An aerosolizable formulation according to claim 1, wherein the
one or more encapsulating materials having a solubility in water of at least 50% of the solubility in water of the one or more flavors to be encapsulated.

3. (canceled)

4. An aerosolizable formulation according to claim 1, wherein in the presence of water the energy of binding of the one or more encapsulating materials with the one or more flavors to be encapsulated is from −2 to −8 kcal/mol.

5. An aerosolizable formulation according to claim 1, wherein in the presence of water the energy of binding of the one or more encapsulating materials with the one or more flavors to be encapsulated is from −3 to −8 kcal/mol.

6. An aerosolizable formulation according to claim 1, wherein in the presence of water the energy of binding of the one or more encapsulating materials with the one or more flavors to be encapsulated is from −4 to −7 kcal/mol.

7. An aerosolizable formulation according to claim 1, wherein water is present in an amount of at least 75 wt. % based on the aerosolizable sable formulation.

8. An aerosolizable formulation according to claim 1, wherein water is present in an amount of at least 90 wt. % based on the aerosolizable formulation.

9. An aerosolizable formulation according to claim 1, wherein the nicotine is present in an amount of no greater than 1 wt. % based on the aerosolizable formulation.

10. An aerosolizable formulation according to claim 9, wherein the nicotine is present in an amount of from 0.01 to 0.6 wt. % based on the aerosolizable formulation.

11. An aerosolizable formulation according to claim 1, further comprising at least one acid.

12. An aerosolizable formulation according to claim 11, wherein the at least one acid is selected from the group consisting of acetic acid, lactic acid, formic acid, citric acid, benzoic acid, pyruvic acid, levulinic acid, succinic acid, tartaric acid, sorbic acid, propionic acid, phenylacetic acid, and mixtures thereof.

13. An aerosolizable formulation according to claim 11, wherein the at least one acid is selected from the group consisting of citric acid, benzoic acid, levulinic acid, sorbic acid, lactic acid, and mixtures thereof.

14. An aerosolizable formulation according to claim 11, wherein the at least one acid comprises citric acid.

15. An aerosolizable formulation according to claim 1, any one of claims 11 to 14 wherein the total content of acid present in the formulation is no greater than 1.0 mole equivalents based on the nicotine.

16. An aerosolizable formulation according to any one of wherein a total content of acid present in the solution is no less than 0.1 mole equivalents based on the nicotine.

17. An aerosolizable formulation according to claim 1, wherein the one or more flavors are selected from (4-(para-) methoxyphenyl)-2-butanone, vanillin, γ-undecalactone, menthone, 5-propenyl guaethol, menthol, para-mentha-8-thiol-3-one and mixtures thereof.

18. An aerosolizable formulation according to claim 17, wherein the one or more flavors comprises menthol.

19. An aerosolizable formulation according to claim 1, wherein the one or more flavors are present in a total amount of no greater than 2 wt. % based on the aerosolizable formulation.

20. An aerosolizable formulation according to claim 1, wherein the one or more flavors are present in a total amount of from 0.01 to 1 wt. % based on the aerosolizable formulation.

21. An aerosolizable formulation according to claim 1, wherein the one or more encapsulating materials comprises one or more cyclodextrins.

22. An aerosolizable formulation according to claim 21, wherein the one more cyclodextrins are selected from the group consisting of substituted (α)-cyclodextrin, unsubstituted (α)-cyclodextrin, substituted (β)-cyclodextrin, unsubstituted (β)-cyclodextrin, substituted (γ)-cyclodextrin, unsubstituted (γ)-cyclodextrin, and mixtures thereof.

23. An aerosolizable formulation according to 21, wherein the one more cyclodextrins comprises a substituted (β)-cyclodextrin.

24. An aerosolizable formulation according to claim 1, wherein the one or more encapsulating materials are present in a total amount of no greater than 12 wt. % based on the aerosolizable formulation.

25. An aerosolizable formulation according to claim 1, wherein the one or more encapsulating materials have a solubility in water of at least 70% of the solubility in water of the one or more flavors to be encapsulated.

26. An aerosolizable formulation according to claim 1, wherein the one or more encapsulating materials have a solubility in water of at least 90% of the solubility in water of the one or more flavors to be encapsulated.

27. An aerosolizable formulation according to claim 21, wherein the aerosolizable formulation contains no flavors that can be encapsulated by the one or more cyclodextrins.

28. A process for forming an aerosol, the process comprising
aerosolizing an aerosolizable formulation comprising:
(i) water present in an amount of at least 70 wt. % based on the aerosolizable formulation;
(ii) one or more flavors to be encapsulated; and
(iii) one or more encapsulating materials;
wherein in the presence of water an energy of binding of the one or more encapsulating materials with the one or more flavors to be encapsulated is from −0.5 to −8 kcal/mol.

29. (canceled)

30. A process according to claim 28, wherein the aerosolizable formulation is aerosolized to form the aerosol at a temperature below 50° C.

31. A process according to claim 28, wherein aerosolizing the aerosolizable formulation comprises applying ultrasonic energy to the aerosolizable formulation.

32. A contained aerosolizable formulation comprising:
(a) a container; and
(b) an aerosolizable formulation comprising:
(i) water present in an amount of at least 70 wt. % based on the aerosolizable formulation
(ii) one or more flavors to be encapsulated;
(iii) one or more encapsulating materials; and
(iv) nicotine;
wherein in the presence of water an energy of binding of the one or more encapsulating materials with the one or more flavors to be encapsulated is from −0.5 to −8 kcal/mol.

33. A contained aerosolizable formulation according to claim 32, wherein the container is configured for engagement with an electronic aerosol provision system.

34. An electronic aerosol provision system comprising:
(a) an aerosolizer for aerosolizing formulation for inhalation by a user of the electronic aerosol provision system;
(b) a power supply comprising a cell or battery for supplying power to the aerosolizer
(c) an aerosolizable formulation comprising:
(i) water present in an amount of at least 70 wt. % based on the aerosolizable formulation
(ii) one or more flavors to be encapsulated; and
(iii) one or more encapsulating materials;
wherein in the presence of water an energy of binding of the one or more encapsulating materials with the one or more flavors to be encapsulated is from −0.5 to −8 kcal/mol.

35. A process for improving the sensory properties of an aerosolized formulation, the process comprising the steps of:
aerosolizing an aerosolizable formulation comprising:
(i) water present in an amount of at least 70 wt. % based on the aerosolizable formulation;
(ii) one or more flavors to be encapsulated; and
(iii) one or more encapsulating materials;
wherein in the presence of water an energy of binding of the one or more encapsulating materials with the one or more flavors to be encapsulated is from −0.5 to −8 kcal/mol.
